(19) United States
(12) Patent Application Publication
(10) Pub. No.: US 2009/0143995 A1
(43) Pub. Date: Jun. 4, 2009

(54) System and Method for Management and Evaluation of Genotyping Data

(76) Inventors: David DINAUER, Brown Deer, WI (US); Donald MUNROE, Brown Deer, WI (US); Zhouhong SHI, Brown Deer, WI (US); Inta KALVE, Brown Deer, WI (US); Ralf WASSMUTH, Dresden (DE); Irina BOEHME, Dresden (DE)

Correspondence Address: CLIENT 51 C/O;GREENLEE , WINNER AND SULLIVAN, P.C, 4875 PEARL EAST CIRCLE , SUITE 200, BOULDER, CO, 80301, US

(21) Appl. No.: 12/204,752

(22) Filed: Sep. 4, 2008

Related U.S. Application Data
(60) Provisional application No. 60/969,940, filed on Sep. 4, 2007.

(51) Int. Cl. C12Q 1/68; C12Q 1/70; G06F 19/00

(52) U.S. Cl. 702/19; 435/6; 435/5

(57) ABSTRACT

Provided are systems and methods for improving efficiency in high throughput genotyping operations by implementing a unique workflow management architecture that permits faster and more accurate determination and evaluation of genotyping and haplotyping, and software to accomplish the same. The system provides a user with a highly-accurate summary and multiple-field breakdown of panels of genotyping data samples for batch approval and batch selection of ambiguous or potentially unique sample sets which can be selected for further analysis. Also provided are tools for evaluating and improving the operation of a genotyping laboratory to maximize the testing and typing of the significant quantities of raw data used in genotyping that are produced in high-throughput laboratory environments.

### CROSS-REFERENCE TO RELATED APPLICATIONS

This application claims benefit of U.S. Provisional Patent Application 60/969,940 filed Sep. 4, 2007, which is hereby incorporated by reference in its entirety to the extent not inconsistent with the disclosure herein.

### BACKGROUND OF THE INVENTION

The present invention relates to a system, method, and computer program product for management and evaluation of genotyping data. More specifically, the invention relates to reducing processing time and increasing efficiency in high throughput typing operations through unique data and workflow management techniques. The invention can be employed with any form of data from which genotyping information can be derived and is useful in particular with sequencing-based typing data, sequence-specific oligonucleotide typing data or both.

The accumulation of genomic information and technology is opening doors for the discovery of new diagnostics, preventive strategies, and drug therapies for a whole host of diseases, including diabetes, hypertension, heart disease, cancer, and mental illness as well as application to transplantation. Many human diseases have genetic components, which may be evidenced by clustering in certain families, and/or in certain racial, ethnic or ethno-geographic (world population) groups. Additionally, genetic components directly influence the immunological response and transplant outcome. To study these genetic factors or generate transplant donor registries, vast numbers of individuals must be genotyped. Repositories of disease related genetic components and their variants are useful in the research and development of health care advancements. Genotyping of non-human animals is further useful in selection of such animals for research and selective breeding applications (e.g., marker-assisted breeding). Genotyping of plants is similarly useful for research, tracing or breeding applications. Genotyping is also useful in the identification of microorganisms, including bacteria, for example for use in the study of infectious disease and for tracing sources of microorganism, e.g., in incidents of food contamination.

Characterization of polymorphism in genes and genetic components can be achieved by many methods, including methods based on hybridization, for example, using sequence-specific oligonucleotides (SSOs), methods based on detection of specific nucleic acid fragments (e.g., RFLP analysis, or selective fragment amplification) and nucleic acid sequencing, particularly DNA (e.g., sequenciy-based typing). The goal of such methods is to define an individual's genotype and/or haplotype.

With the recent advances in biotechnological research enormous amounts of data can be rapidly generated which can be used to determine genotype. For example, most laboratories have access to modern automated DNA sequencing machines that give rise to vast amounts of sequencing data with little hands-on laboratory time. Consequently, enormous amounts of raw sequence data are generated that can be used for genotyping and, for this reason, there is a growing need for automated data processing, including accurately assessing the sequence of bases and the quality of traces obtained for each read, in a process called basecalling.

DNA sequencing involves ordering hundreds of peaks (A, G, C, or T) traditionally separated via size exclusion media, the process can be quite error-prone. Commonly, an automated DNA sequencing machine includes basecalling software as part of the processing software, such as ABI PRISM DNA Sequencing Analysis Software (ABI, 1999), which processes raw trace files, translating them into sequences of bases and assigning an N when resolution is not good. Other DNA sequencing systems have component software for basecalling and assessing the quality of the reads. An example is the MegaBACE 1000 DNA Sequencing System from Amersham Pharmacia/Molecular Dynamics (Sunnyvale, Calif., USA). The purpose of basecalling is to determine the nucleotide sequence on the basis of peaks in the trace. Because traces (and regions within a trace) are of variable quality, the fidelity of “called” nucleotides is also variable. This accuracy for each called base is measured by base quality scores, which evaluate the real sequence accuracy. However, the only method to ensure accurate basecalling for all the bases in a single read is for an individual skilled in the art to visually assess the peaks and manually edit the basecalls.

Typically in highly polymorphic genes, forward and reverse sequencing reactions are performed and analyzed together to offer higher confidence in the basecalling and final genotype or haplotype. Some genotypes and haplotypes require sequence characterization across a large genomic region whereby multiple sequencing reads spanning a large number of bases are required. Furthermore, DNA sequencing is generally performed on both parental chromosomes simultaneously. In high density hyper-polymorphic genetic regions the genotyping results can be ambiguous. A genotyping ambiguity exists when the genotyping results obtained from genotyping data give a choice of multiple combinations.

Genotype ambiguities can arise in determination of genotype by any method. Ambiguous genotypes are exhibited by the fact that the exact sequence of each form of the genetic region cannot be distinguished. For instance, assume there is a gene that has 3 polymorphic positions and that the remaining part of the gene is identical among all individuals. An individual with one copy of TAA and one of ATA would have a genotype of [T/A, T/A, A/A] which is identical to an individual with one copy of TTA and one of AAA. Various methods are known in the art for resolving such ambiguities, in particular DNA sequencing targeting one of the alleles in the ambiguous genotype can be used to resolve such ambiguities.

In high throughput genotyping methods, there is a need for rapidly and efficiently identifying the presence of such ambiguities in genotyping data and efficiently resolving such ambiguities to obtain a final confirmed genotype result.

The typical workflow of sequencing-based typing involves DNA isolation from the tissue or cells from an individual. PCR amplification of the desired genetic region followed by oligonucleotide directed sequencing by synthesis of the amplified region. Samples can be batched together, typically in groups of 96, 384, or 1536, using commercially available reaction plates containing the aforementioned number of wells.

To define the genotype or haplotype of an individual, assembly of multiple sequence reads is required which entails joining the sequences of adjacent reads spanning a large genetic region as well as evaluating the data from single chromosome reads to resolve phasing ambiguities. In most cases, individual sequence data editing is required to resolve discrepant basecalls. Finally, genotype and haplotype assignment is done by comparing the composite sequence to a database of known or previously observed sequences. Various computer implemented methods are known for genotype and haplotype assignment based on such sequencing.

High throughput genotyping methods based on data other than sequencing data follow an analogous workflow and various computer implemented methods are known for genotype and haplotype assignment based on such other data, e.g., SSO-based methods and data.

Many labs use a Laboratory Information Management System (LIMS) to direct the testing of samples and organize the results of any such genotyping methods. However, these complex testing processes demand a separate management and organization system, especially when medium to high sample throughput is undertaken. There is, therefore, a need for computer programs to perform and manage these processes separate from LIMS.

Additionally, to reduce the overall time required for genotype and haplotype assignment, the key is to reduce the manual editing and reviewing time on each data panel. What is therefore needed is an improved workflow and process so that manual review of individual genotype and/or sequence data can be minimized.

### SUMMARY OF THE INVENTION

The present invention relates to a system and method for improving efficiency in high throughput typing operations by implementing a unique workflow management architecture that permits faster and more accurate determination and evaluation of genotyping and haplotyping, and software to accomplish the same. The system provides a user with a highly-accurate summary and multiple-field breakdown of panels of genotype data samples for batch approval and batch selection of ambiguous or potentially unique sample sets for further analysis. Also provided are numerous tools for evaluating and improving the operation of a typing laboratory to maximize the testing and typing of the significant quantities of raw typing data being produced in high-throughput laboratory environments. The system and methods herein are particularly useful for high-throughput sequencing-based typing operations and laboratories which employ raw SBT data as at least a part of the typing operation.

In specific embodiments, data generated by any know method, for example based on nucleic acid sequencing, hybridization to sequence specific oligonucleotide probes, or the detection of specific nucleic acid fragments, can be used to generate the initial summary and determine the quality of the data for genotype determination and to identify genotyping ambiguities. In another specific embodiment, further testing for ambiguity resolution is performed using sequencing-based methods.

In one aspect, the invention relates to a method for evaluating the quality of a plurality of genotyping samples by reviewing an interactive list of genotyping samples. The interactive list relates to genotyping information and at least one pre-selected quality parameter and the interactive list can be displayed by a computer-program product, such as on a computer-usable medium. The plurality of genotyping samples in the list are selected as approved for further use, rejected from further use, or forwarded for further testing to better determine the genotype and at least one quality parameter, the selection being dependent on at least one pre-selected quality parameter.

In a specific embodiment, the method comprises a step of selecting a subset of input samples for listing or interactive listing. Such automated sample selection can be based on a determination of whether or not a particular pre-selected value or range of values of the one or more quality parameters of the data has been met. For example, the selection may exclude samples having no genotype ambiguities from the list.

In an embodiment, the genotype samples forwarded for further testing are samples with genotype ambiguities. More specifically, the genotype ambiguities of the samples are resolved and the samples resubmitted for further evaluation quality. Ambiguity resolution is by any method known in the art capable of providing genotype information. For example, the genotype ambiguities can be resolved through the use of one or more of: sequence-specific oligonucleotide typing, sequencing-based typing, one or more Group Specific Sequence Primers (GSSPs), one or more Specific Sequence Primers (SSPs), one or more of both GSSPs and SSPs.

In another specific embodiment, the genotyping samples are sequence specific oligonucleotide samples or sequencing samples.

In a specific embodiment, the resolution of ambiguities comprises identifying one or more methods for resolving a given ambiguity. In some cases, an identified method may be capable of resolving more than one ambiguity. In a further embodiment, the resolution of ambiguities comprises a step of identifying the fewest number of methods for resolving the greatest number of ambiguities. In specific embodiments, one or more methods are identified for each ambiguity. In yet further embodiments, resolution of ambiguities comprises organizing the further processing of the selected samples for resolving ambiguities.

Organizing the further processing comprises one or more of efficiently organizing samples for further processing to minimize processing time, organizing samples for minimizing movement of sample preparation or of sample processing instrumentation, organizing sample preparation or processing steps to minimize repetitive steps, or organizing sample preparation or processing steps to minimize reagent use. In a specific embodiment, instructions are prepared to accomplish further processes which control or direct the operation of sample preparation and or sample processing instrumentation. Such instructions can be prepared, for example, in the form of one or more plate records, output worklists or scripts for use by such instrumentation. In a more specific embodiment, one or more scripts are prepared to control or direct liquid handling instrumentation to efficiently prepare samples, for example, scripts can be prepared to minimize liquid handler movement during sample preparation for further processing or during such further processing steps. Instructions, output worklists, scripts prepared can be directly employed to control or direct sample preparation or sample processing instrumentation or alternatively, or they can be passed to a LIMS and used by the LIMS to control or direct sample preparation or sample processing instrumentation.

In an embodiment, the resolution of ambiguities comprises identifying one or more methods for resolving a given ambiguity. In some cases, an identified method may be capable of resolving more than one ambiguity. In a further embodiment, the resolution of ambiguities comprises a step of identifying the fewest number of methods for resolving the greatest number of ambiguities. In specific embodiments, one or more methods are identified for each ambiguity. In yet further embodiments, resolution of ambiguities comprises organizing the further processing of the selected samples for resolving ambiguities.

In an embodiment, the resolution of ambiguities comprises one or more steps of organizing the further processing of samples to resolve ambiguities. Such organization comprises organization to increase efficiency of the further processing of sample and may include selections which optimize efficiency (e.g., decrease time for or decrease steps for) of sample preparation or sample processing for accomplishing ambiguity resolution. Organization of samples for further processing can include, among others, grouping samples which are to be processes by the same steps, combination of steps by the same reagents or combination of reagents, or at the same temperature. In a specific embodiment, organization of samples comprises organization of samples in one or more multi-well reaction plate to minimize movement of a liquid handler during preparation of such reaction plate to accomplish further processing.

In a specific embodiment, the resolution of ambiguities comprises determining the type of GSSPs, SSPs or both to use such that the fewest number of GSSPs or SSPs will resolve the greatest number of ambiguities.

In another embodiment, resolution of ambiguities comprises processing samples with at least one ambiguity after GSSP processing through the use of one or more Specific Sequence Primer (SSP) primer. Alternatively, the resolution of ambiguities comprises using a virtual ambiguity resolver when a SSP primer is unavailable, such that the virtual ambiguity resolver generates a virtual result for resolving the at least one ambiguity. In this embodiment, an SSP kit based on the virtual result of the virtual ambiguity resolver is optionally constructed, such that the at least one ambiguity of the genotype sample can be resolved.

The genotyping samples are obtained from any organism or environment of interest. The genotyping samples can be process for any desired genotyping application. For example, the genotyping samples are optionally for immune system receptor genotyping, red blood cell antigen genotyping, bacterial species identification, virus genotyping, or metabolic factor genotyping. In a specific embodiment, the genotyping samples are HLA genotyping samples.

In another aspect, the invention relates to a method for managing and evaluating genotyping data, including receiving a plurality of data from which genotype can be determined from a plurality of genotype samples and generating a worklist from the plurality of data from a plurality of samples, wherein the worklist includes at least information identifying each sample. The sample data is processed to determine genotype and at least one quality parameter of the genotype determination. A summary is displayed of at least one genotype and at least one quality parameter of the sample data of at least a portion of the plurality of samples for evaluation by a user. Optionally the worklist generation is by importing a worklist from a Laboratory Information Management System (LIMS). Optionally, one or more quality parameters of the sample data are imported from a system or instrument or device which generates the data used for typing, e.g., a DNA sequencer.

The quality parameter is any parameter that provides information about data quality. For example, the parameter may be signal-to-noise ratio, basecall records, quality value of the typing result, and mismatching counts. In an embodiment, the quality value of the typing result comprises a genotyping ambiguity. In this embodiment, the genotype ambiguities are optionally further processed, such as processed to at least partially resolve one or more ambiguities.

Any of the methods provided herein may resolve genotyping ambiguities by genotyping procedures known in the art, including but not limited to the use of sequence-specific oligonucleotide typing or sequencing-based typing. Resolution of ambiguities by sequencing based typing may comprise the use of one or more Group Specific Sequence Primers (GSSPs), one or more Specific Sequence Primers (SSPs), or both.

In an embodiment of the methods of the invention, ambiguity resolution comprises identifying one or more methods for resolving ambiguities and optionally generating instructions for carrying out the further processing, particularly instructions which provide for efficient sample preparation and or processing for carrying out the further processing.

In an embodiment of the methods of the invention, ambiguity resolution comprises determining the type of GSSPs, SSPs or both to use such that the fewest number of GSSPs or SSPs will resolve the greatest number of ambiguities.

In specific embodiments, any of the methods provided herein optionally further measure the time required for processing the genetic sequence data, analyzing the resulting typing and related information, and making a usability determination to look for problems, identify delays or both in the high-throughput genetic sequencing process.

In another aspect, the invention relates to a method for determining the quality of high-throughput data from which genotype can be determined of a plurality of samples. In this embodiment, data of a plurality of the genotype samples is processed to determine genotyping information and at least one quality parameter of the data. A summary is displayed of the genotyping information and the at least one quality parameter for at least a portion of the plurality of samples processed. In an embodiment, the Summary includes all of the processed samples. The summary of the genotyping information and at least one quality parameter is analyzed to determine the usability of the displayed samples for determining genotype at the same time point.

Any of the methods provided herein, are optionally carried out on a computer-program product embodied in a computer-usable medium.

Also provided are products, such as a computer program product embodied on one or more computer-usable mediums, comprising computer instructions for carrying out one or more steps of the method of this invention. In an embodiment, the computer program product is employed for determining the quality of high-throughput data which can be used for determining genotype of a plurality of samples. More specifically, the program comprises computer instructions for processing the data for a plurality of samples to determine typing information and at least one quality parameter of the data and displaying the typing information and at least one quality parameter for at least a portion of the plurality of samples in a single view, such that a user can analyze and make determinations of the usability of the samples for genotyping without requiring analysis of individual sample typing data. The computer program optionally comprises computer instructions for selecting a subset of input samples for listing or display or interactive listing or display. Such automated sample selection can be based on a determination of whether or not a particular pre-selected value or range of values of the one or more quality parameters of the data has been met. For example, the selection may exclude samples having no genotype ambiguities from the list or display or interactive list or display.

In another embodiment, the computer program further comprises computer instructions for measuring the time required for processing the data, analyzing the resulting genotyping and at least one quality parameter, and determining usability to look for problems, identify delays or both in the high-throughput genetic sequencing process. The computer program optionally has computer instructions for further processing at least one sample selected by a user for further analysis, wherein the further processing comprises resolution of at least one genotype ambiguity. In a specific embodiment, computer instructions are provided for identifying one or more processes for resolving the ambiguity, such as by a process that comprises sequencing-based typing or typing by sequence-specific oligonucleotides. In a more specific embodiment, the computer program comprises computer instruction for identifying one or more GSSPs for resolution of the ambiguity, or identifying one or more SSPs for resolution of the ambiguity.

In another embodiment, the computer program has computer instructions for generating a script to provide instructions for further processing the one or more samples by the one or more identified processes for resolving the ambiguity, such as a script that is for use by a liquid handler.

A specific embodiment of the invention provides a method for determining the quality of high-throughput genetic sequence data of a plurality of sequencing samples comprises: processing genetic sequence data of a plurality of sequencing samples to determine typing information and at least one quality parameter of the genetic sequence data; displaying a summary of the typing information and the at least one quality parameter for the plurality of samples; and analyzing the summary of the typing information and at least one quality parameter to determine the usability of a substantial majority of the plurality of samples at the same time point.

In a further specific embodiment, the invention provides a method for managing and evaluating HLA typing data comprises: receiving a plurality of sample sequence data from a plurality of samples; constructing a worklist from the sample sequence data, wherein the worklist includes information identifying each sample; processing the sample sequence data to determine the HLA type and at least one quality parameter of the HLA type determination; and displaying a summary of the HLA type and at least one quality parameter of the plurality of sample sequence data for evaluation by a user.

In a further specific embodiment, the invention provides a method for evaluating the quality of a plurality of HLA typing samples comprises: reviewing an interactive list of HLA samples, wherein the interactive list comprises HLA typing information and at least one pre-selected quality parameter, and wherein the interactive list is displayed by a computer-program product embodied on a computer-usable medium; and selecting a plurality of HLA samples in the list as approved for further use, rejected from further use, or forwarded for further testing to better determine the HLA type and at least one quality parameter, the selection being dependent on at least one pre-selected quality parameter.

In another specific embodiment, the invention provides a computer-program product embodied on one or more computer-usable mediums for determining the quality of high-throughput genetic sequence data of a plurality of sequencing samples comprises: computer instructions for processing genetic sequence data for a plurality of samples to determine typing information and at least one quality parameter of the genetic sequence data; and computer instructions for displaying the typing and at least one quality parameter for the plurality of samples in a single view, such that a user can analyze and make determinations of the usability of a substantial majority of the samples without requiring analysis of individual sample typing information.

One or more methods of the invention can be employed in an immunodiagnostic method for assigning HLA types to two or more samples.

In a specific embodiment, the methods herein identify reagents products for carrying out genotype resolution. In a more specific embodiment, the methods include a step of assessing the availability of reagents on-site and optionally provide a report indicating reagent availability or optionally initiate ordering of reagents, for example by generating an order for one or more reagents for transmission to a vendor. This step can optionally be expanded to track on-site reagent inventory to optionally generate alarms when such inventory reaches a pre-selected minimum level or generate a reagent order for transmission to a vendor.

In a specific embodiment, the methods herein track the status of each sample and/or a panel of samples. The status can include the owner and or level of user authorization of a sample and/or panel and time stamp to indicate the first time the panel was loaded and/or time stamps for subsequent loading or review of samples and/or panels. Each sample and/or panel can be designated with a status of “pending” or “reviewed and approved.” Time stamps can be provided for status changes particularly for change of status to reviewed and approved. Any samples and/or panels can be locked by the owner and or by an appropriately authorized user. The methods herein can track time stamps on each sample and/or panel to provide information regarding efficiency of sample/panel processing to review and approval, for example.

In an embodiment, a due date can be assigned to each sample and/or panel. Such due dates can be tracked for each sample or panel and a warning to users can be provided at selected times prior to the due date. The turn-around time can further be used to indicate productivity. The warning may be a visual indicator or an email message. Samples can also be assigned a priority with a selected prioritized due date.

Other aspects and embodiments of the invention will be appreciated on review of the drawings and detailed description.

### BRIEF DESCRIPTION OF THE DRAWINGS

FIG. 1 is a flow chart of a software system for processing and evaluating data used to determine a genotype, according to one aspect of the invention;

FIG. 2A is a flow chart of a super high throughput (SHTP) workflow according to an embodiment of the invention that expands the highlighted portion of FIG. 1; FIG. 2B is a flow chart of batch mode workflow for a worklist generated from SSO experiments;

FIG. 3A is a flow chart of a batch mode workflow corresponding to the panel overview step of FIG. 1 for reviewing a panel that has been loaded into the software system for processing, according to an embodiment of the invention; FIG. 3B is a flow chart for a worklist generated from SSO experiments;

FIG. 4A is a flow chart of a batch mode workflow corresponding to the panel load step of FIG. 1 for importing a worklist into the software system for processing, according to an embodiment of the invention; FIG. 4B is a flow chart for a worklist generated from SSO experiments;

FIG. 5 is a flow chart of a batch mode workflow corresponding to the panel review step of FIG. 1, according to an embodiment of the invention.

FIG. 6 is a screen capture image of a visual display of the panel load step of FIG. 1, according to an embodiment of the invention;

FIG. 7 is a screen capture image of a visual display of the panel overview step of FIG. 1, according to an exemplary embodiment of the invention;

FIGS. 8, 9 and 10 are flowcharts of a workflow for implementing a GSSP to resolve an ambiguity in a sample, according to an exemplary embodiment of the present invention;

FIG. 10 is an illustration of a panel for use in implementing a GSSP to resolve ambiguity in a sample, according to an exemplary embodiment of the present invention;

FIG. 11 is an illustration of an exemplary embodiment of a control chart of a lab view for reviewing and organizing the activities of a high-throughput genetic sequence lab; invention;

FIG. 12 is a flow chart illustrating the access a regular user is permitted to certain features of the software system, according to an exemplary embodiment of the present invention;

FIG. 13 is a flow chart illustrating the access a supervising user has to certain features of the software system, according to one aspect of the present invention;

FIG. 14 is an illustration of a visual representation of a panel view used to implement and analyze the results of GSSP to resolve an ambiguity in a sample, according to an exemplary embodiment of the invention; and

FIG. 15 is an illustration of a layout print used for setting up a GSSP implementation to resolve an ambiguity in a sample, according to an exemplary embodiment of the present invention.

### DETAILED DESCRIPTION OF THE INVENTION

Provided are systems and methods for improving efficiency in genotyping operations by implementing a unique workflow management architecture that permits faster and more accurate determination and evaluation of genotyping and haplotyping, and software to accomplish the same. The system provides a user with a highly-accurate summary and multiple-field breakdown of panels of sequence samples for batch approval and batch selection of ambiguous or potentially unique sample sets for further analysis. Also provided are numerous tools for evaluating and improving the operation of a genotyping laboratory to maximize the testing and typing of the significant quantities of raw data being produced in high-throughput laboratory environments, such as SBT data or other data that provides information useful for genotyping (e.g., probing of hybridization with DNA or fragments thereof, such as by SSO). The input into the workflow and methodologies disclosed herein can be of any format and arise from any number of different techniques. For example, in an aspect the input worklist is from high throughput sequencing-based typing (SBT). In another aspect, the input worklist is from sequencing specific oligonucleotides (SSO). Similarly, any other data for genotyping is compatible with the processes and systems disclosed herein, wherein the improved methodology reduces processing time and increases efficiency, thereby providing faster and more reliable genotyping results.

Glossary

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this invention is related. The following terms are defined for purposes of the invention as described herein.

Sequencing File: defines the data file which contains sequencing raw data.

Sequencing Data Analysis: a data analysis process including import of sequencing raw data, making basecall to sequence base, aligning sequence bases, and typing.

Sequencing Raw Data: sequencing trace files from the manufacturers' sequencing machines such as Amersham Biosciences, Applied Biosystems, Beckman Instruments, and LI-COR Life Sciences.

Sequencer: sequencing machine from Amersham Biosciences, Applied Biosystems, Beckman Instruments, or LI-COR Life Sciences.

Allele: a particular form of a genetic locus, distinguished from other forms by its particular nucleotide sequence.

Polymorphic site: a nucleotide position within a locus at which the nucleotide sequence varies from a reference sequence in at least one individual in a population. Sequence variations can be substitutions, insertions or deletions of one or more bases.

Phased: as applied to a sequence of nucleotide pairs for two or more polymorphic sites in a locus, phased means the combination of nucleotides present at those polymorphic sites on a single copy of the locus is known.

Gene: a segment of DNA that contains all the information for the constitutive or regulated biosynthesis of an RNA product, including promoters, exons, introns, and other untranslated regions that control expression.

Genotype: an unphased 5′ to 3′ sequence of nucleotide pair(s) found at one or more polymorphic sites in a gene on a pair of homologous chromosomes in a diploid individual or on a chromosome where the individual is not diploid.

Genotyping: a process for determining a genotype of an individual or information that may in turn be used to determine genotype. The processes and systems provided herein are compatible with any genotyping application. Examples of genotyping applications include, but are not limited to, HLA, immune system receptors (e.g., KIR, MICA), red cell Ag (RHD, ABO), bacterial species identification (e.g., Legionella), virus genotyping (e.g., HCV, HIV), metabolic factors (CYP450, CYP3A5). See, e.g., Cuevas JM et al. (2008) Genetic Variability of Hepatitis C Virus before and after Combined Therapy of Interferon plus Ribavirin. PLoS ONE 3(8): e3058. doi:10.1371/journal.pone.0003058; M. Scaturro et al. Comparison of Three Molecular Methods Used for Subtyping of Legionella pneumophila Strains Isolated during an Epidemic of Legionellosis in Rome JOURNAL OF CLINICAL MICROBIOLOGY, October 2005, p. 5348-5350; Helene Polin et al. “Effective molecular RHD typing strategy for blood donations” Transfusion 47(8), 1350-1355 (2007). Genotyping is used broadly and, in an aspect, includes the term haplotyping.

Haplotype: a member of a polymorphic set, e.g., a sequence of nucleotides found at two or more polymorphic sites in a single chromosome of an individual. This also refers to the collection of polymorphic sites within a gene or between two or more genes on a single chromosome.

Plate record: a file containing sample information that can be imported into a sequencer.

“Script” refers to information output from the methods of the present invention and more specifically, to information that can be used in subsequent protocols useful in obtaining genotyping information. For example, the script can control the movement of an automated liquid handler, robotic arm or the like for transferring samples, applying reagents or other activity related to resolution of ambiguities for genotyping.

Script file for Liquid Handler: a file containing control information for liquid handlers from manufacturers such as Tecan, PerkinElmer.

In certain embodiments, an interactive list is created and/or displayed. Such a list can, for example, comprise typing information for one or more (typically more than one) sample and one or more quality parameters, typically for each sample in the list. The list is interactive in that it enables a user to flag, tag, or otherwise identify or select, reject or delete, one or more items in the list. For example, an item in the list can be selected for further processing or can be rejected as defective or otherwise unusable for genotyping. The list is typically displayed for review by one or more users. User review is optionally controlled by a user authorization scheme which creates a user authorization hierarchy (or levels of authorization) in which certain users are authorized to take only certain actions with the list while other users may be authorized to take any or all available actions. For example, a two tier user authorization hierarchy comprising a regular user and a supervisory user can be established in which the regular user can view the list and make tentative selections, but wherein one or more of the selections made must be reviewed by the supervisory user prior to finalizing the selection and taking the action selected.

“Sample” refers to any material containing nucleotides for which genotyping is desired. A sample may be obtained from a biological material. A sample may be obtained from an environment for which testing of the presence of absence of a genotype is desired.

“Selecting” refers to an examination of samples to classify samples into one or more categories that will dictate subsequent activity for the selected samples. A sample selected for “further testing” refers to a sample for which further information about the sample is desired. For example, a sample having an ambiguity is desirably selected for further testing to resolve the ambiguity and thereby better determine the genotype. The selecting may be performed by a human operator or user, be automated such that a particular selection is made depending on preset values or value ranges provided to the system by the user, or a combination of both human and automated selection. For example, samples falling outside a preselected signal to noise range may be automatically rejected. Similarly, samples having a unique ambiguity solution may be selected to not be displayed to a user who may be performing the selection step. A human user may then be faced with fewer samples for which their explicit selection analysis is required.

“Cherrypicking” refers to implementing an efficient process for further sample processing and particularly for ambiguity resolution. For example, source plates may be positioned relative to a destination plate to minimize travel of a liquid handler head. Similarly, various samples may be grouped according to the subsequent resolution experiments to further increase efficiency, such as by grouping samples requiring identical reagents. In addition, the process of cherrypicking includes implementing the least number of subsequent resolution experiments while achieving the maximum number of ambiguity resolution. Accordingly, cherrypicking is used broadly to refer to any of these one or more steps that decrease time or increase efficiency for subsequent resolution-type experiments.

“Worklist input” refers to data or other ordered information that is input to the process of the present invention. The data is useful for determining genotype information of a sample. For example, the worklist may correspond to the output from an automated sequencer (e.g., such as for Sequence Based Typing), from SSO, or any other technique that provides information useful for genotyping. The specific format of the worklist does not impact the subsequent workflow and outputs as provided herein. “Worklist output” refers to data or other ordered information that is provided by a method or system disclosed herein. For example, worklist can include one or more of instructions, lists, plate records, scripts. A worklist input to a method herein is typically different from a worklist output of that method.

“Improved efficiency of genotyping” refers to various means for assessing any one of the following: decreased time for processing or resolving genotype; increased sample output per unit time; increase in sample accuracy per unit time; or decreased reagent use per sample genotyped. One of ordinary skill in the art will appreciate that efficiency can be assessed in ways other than those specifically exemplified herein. In a preferred embodiment, efficiency improvement is by an at least 10%, or more preferably an at least 25%, decrease compared to conventional genotyping in any of the one or more parameters used to assess efficiency. Efficiency of genotyping can be improved in the methods herein by any one or more of the following: minimizing processing time, organizing samples for minimizing movement of sample preparation or of sample processing instrumentation, organizing sample preparation or processing steps to minimize repetitive steps, or grouping samples which are to be processed by the same steps, combination of steps by the same reagents or combination of reagents, or at the same temperature. In a specific embodiment, organization of samples comprises organization of samples in one or more multi-well reaction plate to minimize movement of a liquid handler during preparation of such reaction plate to accomplish further processing.

A computer program product of this invention can be provided in a computer software system which optionally includes a computer program for determining a genotype from data, such as data from nucleic acid sequencing, nucleic acid probe hybridization or detection of specific nucleic acid fragments, which can be employed to determine genotype. The computer program for determining genotype from data can be a commercially available commuter program, such as uTYPE® HLA Sequencing Software (Invitrogen, Carlsbad, Calif.) or Assign SBTTM software, or RELI™ SSO Pattern Matching Program (PMP) Software. The computer software system may also comprise one or more computer programs comprising computer instructions for data collection and/or sorting, for data quality review, for data analysis, for automated sample preparation (e.g., liquid handler control software), for searching one or more databases to retrieve information there from, and/or for generating reports containing data and/or genotype results.

The examples below are given so as to illustrate the practice of this invention. They are not intended to limit or define the entire scope of this invention. The reagents employed in the embodiments below are commercially available or can be prepared using commercially available instrumentation, methods, or reagents known in the art. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

The goal of one aspect of the invention is to increase the efficiency in genotyping by optimizing processes and workflow, reducing human processing time and potential for human error, as well as ambiguity reduction of samples. In one embodiment, the process is for Human Leukocyte Antigen (HLA) high-resolution typing. The process and systems provided herein are compatible with worklists generated by a variety of experimental techniques that are used for genotyping. For example, the worklist may be generated from sequence information (e.g, sequence based typing (SBT)) or from probes to DNA fragments (e.g., sequence specific oligonucleotides (SSO)).

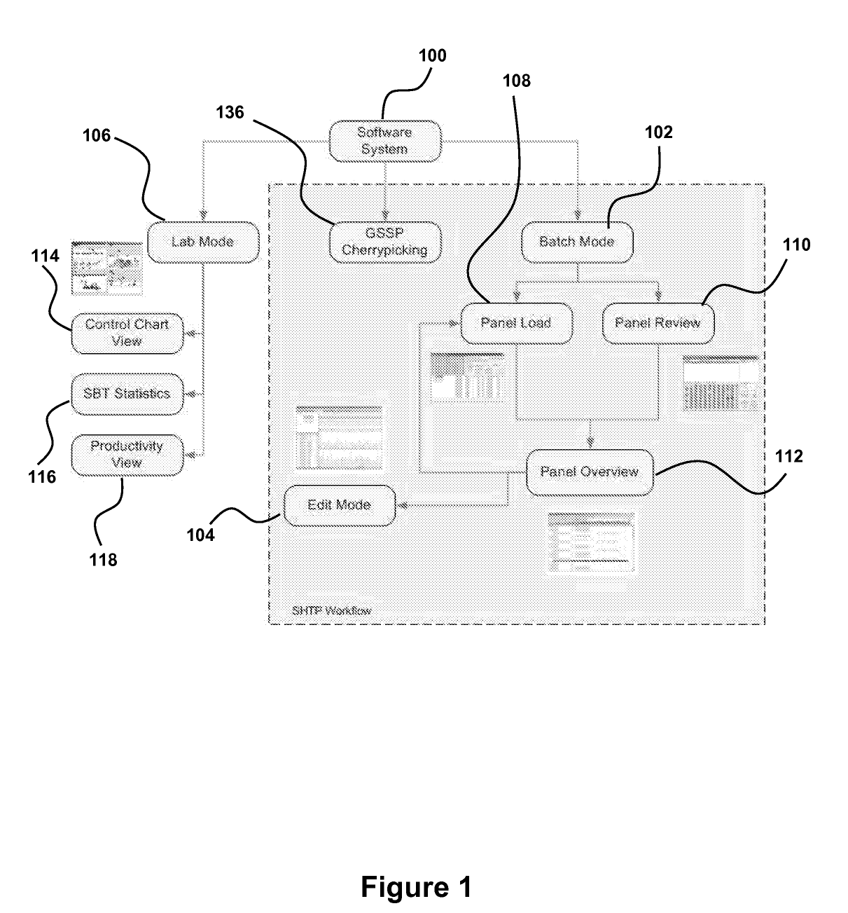

Referring to FIG. 1, the paradigm described herein links experiments used for determining genotype (such as by, for example, SBT or SSO) to two new modes of operation suited to high throughput or super high throughput operation of a genotyping lab. SBT typing analysis is provided by uTYPE and similar programs today and is referred to herein as “Edit mode” 104. uTYPE v2.0, A HIGH THROUGHPUT HLA TYPING SOFTWARE THAT DOES NOT COMPROMISE ACCURACY. Joel Shi, Mary Parlow, Inta Kalve, Tina Agostini, Donald Munroe, Human Immunology 2006 vol 67 supplement 1 pg S147. Similarly, other Edit Modes are available for other experimental systems, such as for SSO type methodologies. Accordingly, Edit Mode 104 refers to typing analysis software as known in the art, including but not limited to, ASSIGN Software TISSUE ANTIGENS 2004 64:556-565, HLA Factura Software, TISSUE ANTIGENS 2003 53:275-281, SBTEngine Software HUMAN IMMUNOLOGY 2004: 65, Supplement1 p S89. Two new modes, “Batch Mode” 102 and “Lab Mode” 106, offer extended functionality, standardized workflow, and dramatic improvements to lab productivity, efficiency and throughput without sacrificing quality or the application genotyping subject matter expertise to mass typing operations.

Batch Mode and Lab Mode each include several work screens or “views” which optimize data presentation and operator decisions to the needs of a high throughput SBT lab. The functions include: computer-aided ambiguity resolution workflow management and quality/productivity metrics for end users; panel tracking of samples at critical points in the workflow; ability to combine panels and fractionate samples into new panels for subsequent data generation and analysis; combining typing information from various methodologies; and donor selection by comparing data results. Batch results flag ‘wanted’ as well as quality errors etc. new alleles, and genotype likelihood scores based on historical information (linkage disequilibrium).

Generating Worklist Input

In one embodiment, the software system is used in automated high-throughput workflow and productivity enhancement for genotyping by synthesis-based nucleic acid sequencing. The invention, as discussed is not limited to synthesis-based nucleic acid sequencing, but can be used for other information such as SSO or other techniques useful in the art of genotyping. The system tracks batch workflows and can drive sample handling, assay setup, and data analysis; review of genotype data; approval of genotyping results; tracking of subsequent follow-up testing for ambiguity resolution; output of quality and productivity metrics.

In this example of HLA-DRB1 high-throughput genotyping, several methods and reagents for generating sequence data for HLA-DRB1 have been described. For this example an approached described by Sayer et al. Tissue Antigens 2001:57:46-57 will be used. In brief, genomic DNA is isolated from whole blood from using a commercially available method (PureLink 96 Genomic DNA kit Invitrogen, Carlsbad, Calif.). The DNA is then subjected to PCR amplification by oligonucleotides specific for exon2 of the HLA-DRB1 gene. PCR products are prepared for dideoxy-terminator sequencing by incubation with exoSAP-IT (USB) to eliminate interaction from excess PCR amplification primers and dNTPs. Two cycle sequencing reactions are performed for each amplified sample in forward and reverse directions by HLA-DRB1 specific oligonucleotides. Dideoxy nucleotides chemistry (DYEnamic ET terminators Amersham GE) is used to label the sequencing fragments. Elimination of excess dye-labeled terminators is accomplished by ethanol precipitation. The sequencing fragments are analyzed on an automated sequencer (ABI) and the resultant output files are saved to a computer network location. This output file (as well as output files applicable for any other relevant technique besides sequencing, such as by probing or SSO, for example) is used by the processes and systems disclosed herein to efficiently and accurately determine a genotype for a characteristic of interest.

Worklist Input Processing

The workflow steps described herein are outside the physical laboratory processing of the samples. The features of the software are implemented after DNA isolation. The process is initiated by a Laboratory Information Management System (LIMS) via typing requests. Worklists are generated from LIMS defining the grouping of samples, such as whole blood samples for DNA isolation processing. For example, in a 96-well system, 95 whole blood samples may be purified, including a previously genotyped positive control (e.g., a “control sample”), along with optionally a “negative control” (such as water only for buffer/reagent negative control). Isolated DNA is stored in a 96 well sample receptacle, referred to as a panel. A portion of the DNA from the panel is processed through the sequencing process described above and the resultant ABI output sequence files are made available on the network storage system. Analysis of the sequences is done in the software where the user imports the LIMS worklist containing the Panel name, sample names and due date for genotyping output.

The processes and systems described herein accept inputs from any LIMS, referred to herein as a “worklist input”. In an aspect, the worklist input includes panel name, sample names, and typing due dates. The output from the invention includes panel name, sample names, and genotypes which can be read by a LIMS. The invention generates and manages traceability of worklists, all raw data files, user basecalling/editing data, time stamps, and associated quality metrics. Outputs of this information are can be provided in different formats as desired to monitor genotyping quality and laboratory productivity. Additional input from LIMS, users or supervisors can also be incorporated that focuses the process on targeting potentially useful or rare genetic information such as specific sequence motifs, alleles, genotypes, and haplotypes. This feature allows enhanced development of the population database.

Systems, methods, and computer program products are described herein to address these and other needs. In accordance with one embodiment, a method is described for management of samples and data for genotyping (such as a worklist arising from sequence-based procedures), as illustrated in FIG. 1. The processes and workflow are centered on features that are available in a Batch Mode from the software system.

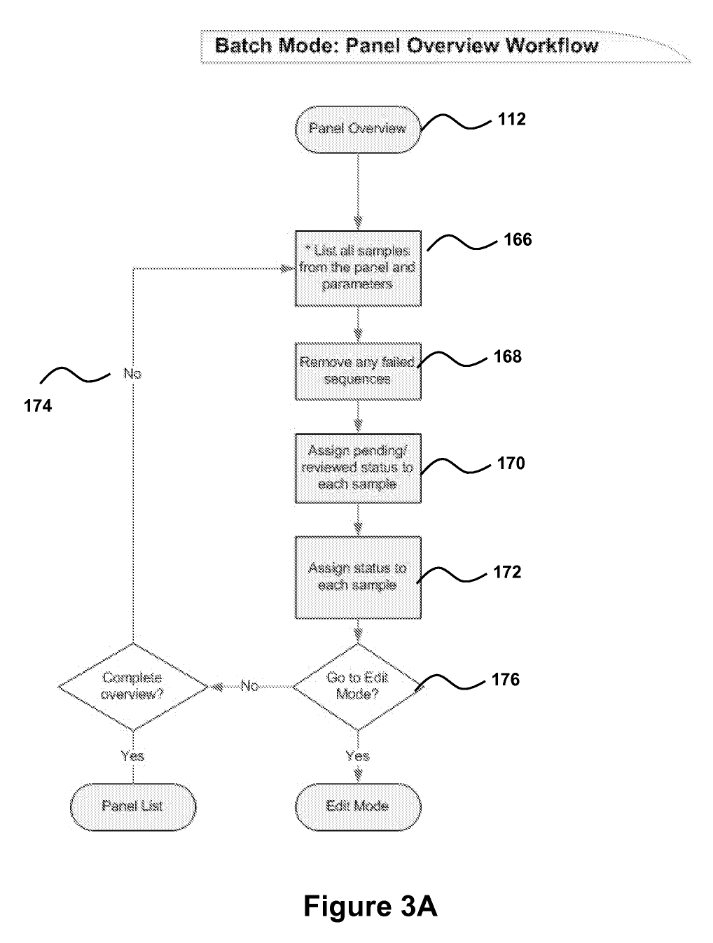

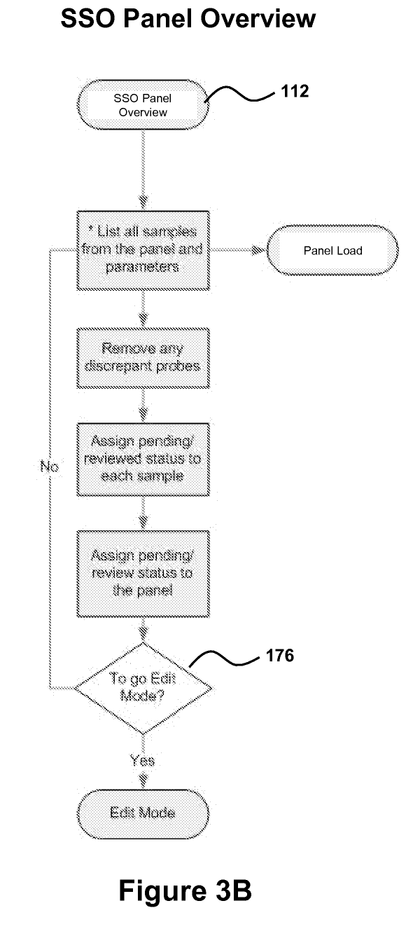

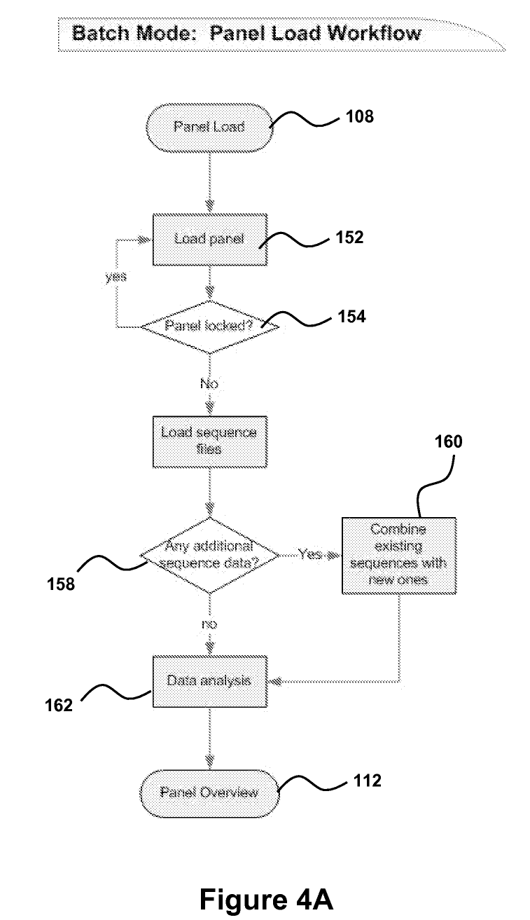

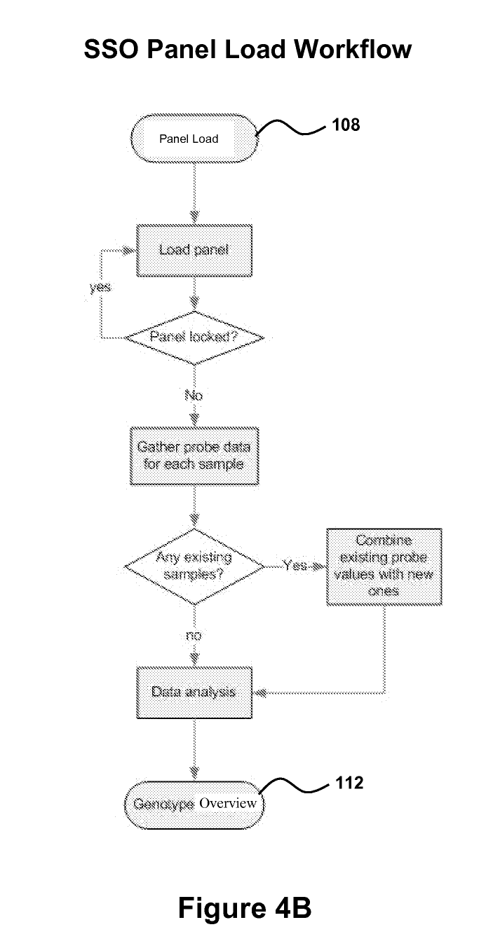

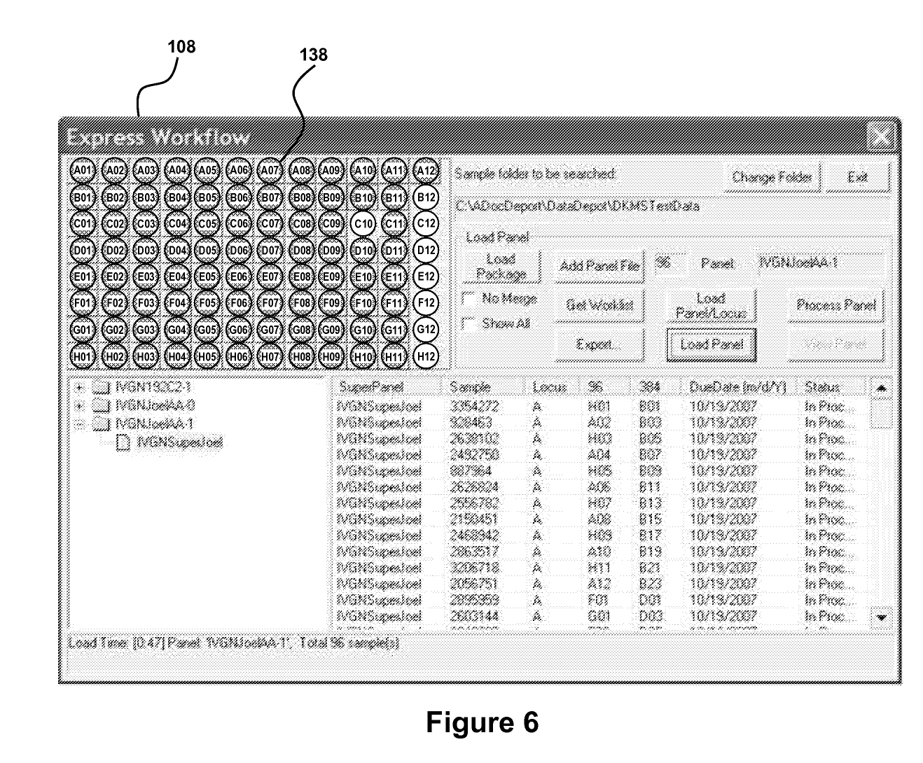

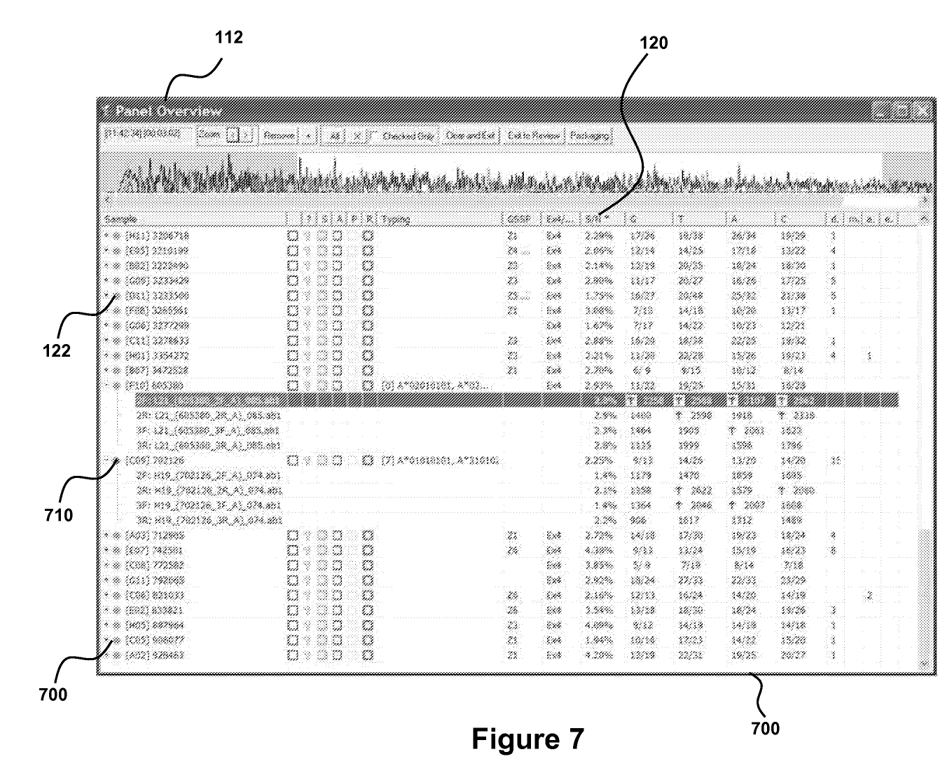

The features of the software system 100 are divided into three categories, or Modes, as illustrated in FIG. 1. Batch Mode (BM) 102 provides methods for high-throughput data analysis. It includes two panel views, the Panel Load 108 and the Panel Review 110. The Panel Load 108, illustrated in FIG. 4, provides a user a way to import a worklist and conduct further sequence data processing. FIG. 4A refers to a worklist arising from sequence-based procedures, whereas FIG. 4B refers to a worklist arising from probe-based procedures (e.g., SSO). The workflow for the Panel Load 108 is set forth in FIG. 4 (and also the illustration of FIG. 6), and illustrates how the Panel Load 108 transitions to the Panel Overview 112 (FIG. 4A is for worklist inputs containing sequence-type data; FIG. 4B for worklist inputs containing probe type information (e.g., SSO)). The Panel Load 108 then enters Panel Overview 112, shown in the workflow of FIG. 3 and the illustration of FIG. 7, which has a summary of samples from the worklist. From Panel Overview 112, a user can review the worklist and make a quick decision to submit the worklist or mark any samples that need further study (e.g., as indicated by the columns in FIG. 7 labeled “s” (submit for approval); “a” (approve); “p” (pending); “r” (repeat—failed sample). The user can then go back to Panel Load 108 for additional worklist processing or go to an Edit Mode 104 for further study.

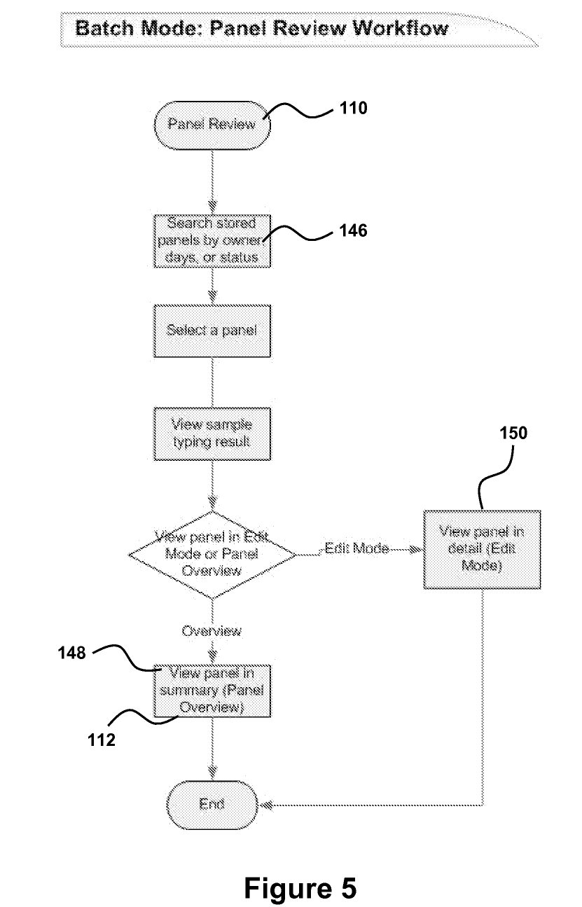

BM Panel Review 110, shown in the workflow of FIG. 5, provides a review for all processed worklists through query. The user can further enter BM Panel Overview 112 through BM Panel Review 110.

Edit Mode 104 is the regular sequence analysis, as known in the art. (see, e.g., Assign: a complete software package for allele assignment and quality control of DNA sequencing based typing David C. Sayer and Damian M. Goodridge, Human Immunology, Volume 63, Issue 10, Supplement 1, October 2002, Page S9).

Lab Mode 106 provides a status overview of the typing activities in a given organization. It contains various views, especially a control chart view 114, a SBT statistics view 116, and a productivity view 118.

The Batch Mode 102 is operated on a sample unit defined as a “panel” (not shown). A panel is a subset of a more general term of worklist, and contains at a minimum categories for locus, sample, well, and panel name. A worklist is defined as a list of samples that will be processed by a certain procedure. Construction of worklists contain sample names, panel name and a typing due date. A panel is a collection of sample names from one or multiple loci, for a certain number of wells on a plate (e.g., 96, 384 or 1536 well tray layout). A “super panel” is defined as a worklist, like a panel, in a 384 well tray layout. It also can be defined as other well tray layout such as 1536 well tray layout. A super panel therefore can contain multiple panels.

In HLA Sequence typing, a 96 well tray typically is used by a sequencer, such as ABI 3730xl (Applied BioSystems Hayward, Calif.). A 384 well tray is used for sample preparation and cleanup. Using panel and super panel makes it possible to track samples between various SBT typing steps.

A sample is identified by a tracker. At a minimum, a “tracker” contains a sample ID, a panel ID, a super panel ID, a 96 well number, and a 384 well number if present.

An aspect of the present invention relates to the concept of providing different authorization levels to control what portions of the process are available to a user. For example, a user or the authorization thereof, can be categorized into two types, regular and supervisor. A regular user is designated for those whose responsibility is to initially load a panel, process the panel, and submit the panel for approval. A supervisor user may review the submitted panel and approve the panel. An approved panel typically is transferred to a LIMS for clinical processes such as report or archival. A regular user in the said software system and in the present invention may also be referred to a technician user or simply a user.

The sample sequence data files from a sequencing instrument are searched and loaded into the said software system as a worklist input. Similarly, sample probe data files from a probing instrument (e.g., an SSO-type instrument) are searched and loaded into the said software system as a worklist input. The data files may be located in a networked storage device. The software system analyzes the data and gives typing results. A viewing and editing window will then be displayed to a user, such as the BM Panel Overview 112 depicted in FIG. 7. The typing results along with editing information and raw data are also stored in a database and storage folders.

The software system 100 may display the overall results and at least one quality parameter 120 related to the typing in a summarized window 700 without the capability for editing, such as the BM Panel Overview 112 shown in FIG. 7. The parameters may include the noise and signal that characterize the sequence electropherogram (indicated by “S/N” 120 in FIG. 7), quality value as determined by statistics or curve shapes, basecall records, and mismatching counts. Similarly, the value for each base column (labeled G T A C) provides a measure of signal value and is, therefore, a quality parameter. The additional columns labeled d, m, a, e may also provide a measure or quality and so can be considered a quality parameter. In addition, quality parameter may refer to the presence or absence of a genotyping ambiguity for a sample, including whether the ambiguity has one unique match such as a GSSP that is capable of resolving the ambiguity. The said summarized window 120 may be a display window to show the parameters or a file to be loaded by other third party software systems for viewing the parameters. Further, the said display window 120 may show parameters in a color-coded visual assistant 122 way to highlight the analysis. Panel overview 112 provides a convenient, fast and efficient platform for a user to identify potential ambiguities and suggested GSSP protocols for resolving ambiguities, for example. In an aspect, samples not requiring further analysis (e.g., those with a full green dot as identified by 122) are optionally not displayed, further increasing efficiency. In an aspect, a user provides a selected range or cut-off value for various quality parameters to provide further automated handling of the analysis step that occurs with this panel overview window.

The said software system 100 further provides a way to retrieve a processed worklist for review, further analysis and editing. The results of the further analysis and editing are stored in the database and storage folders with history information.

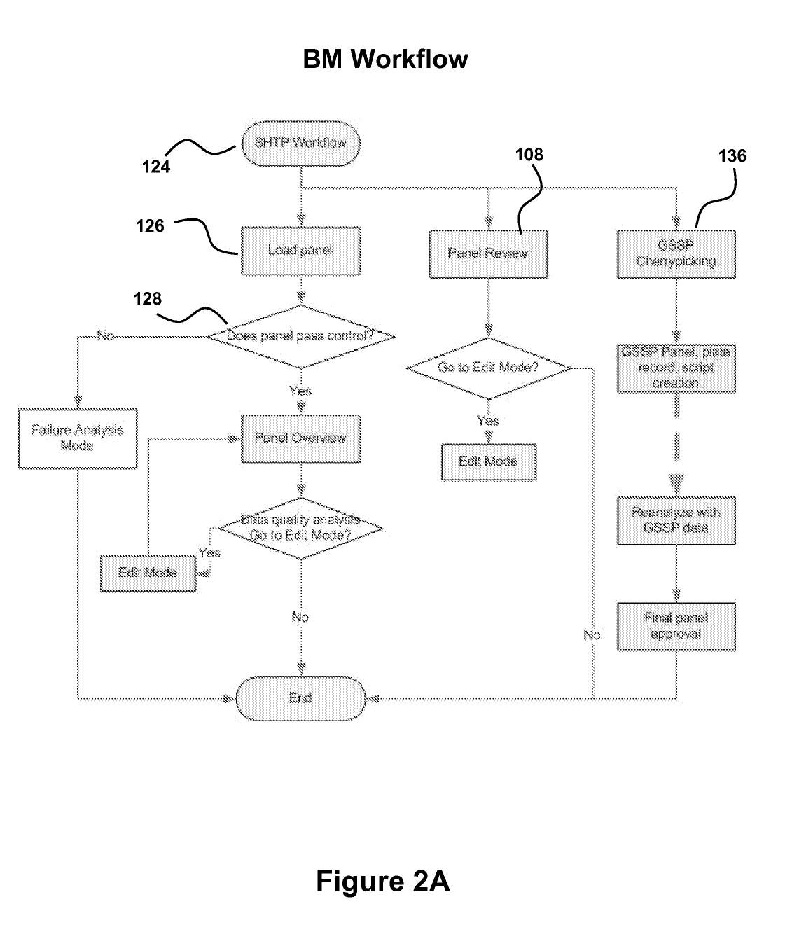

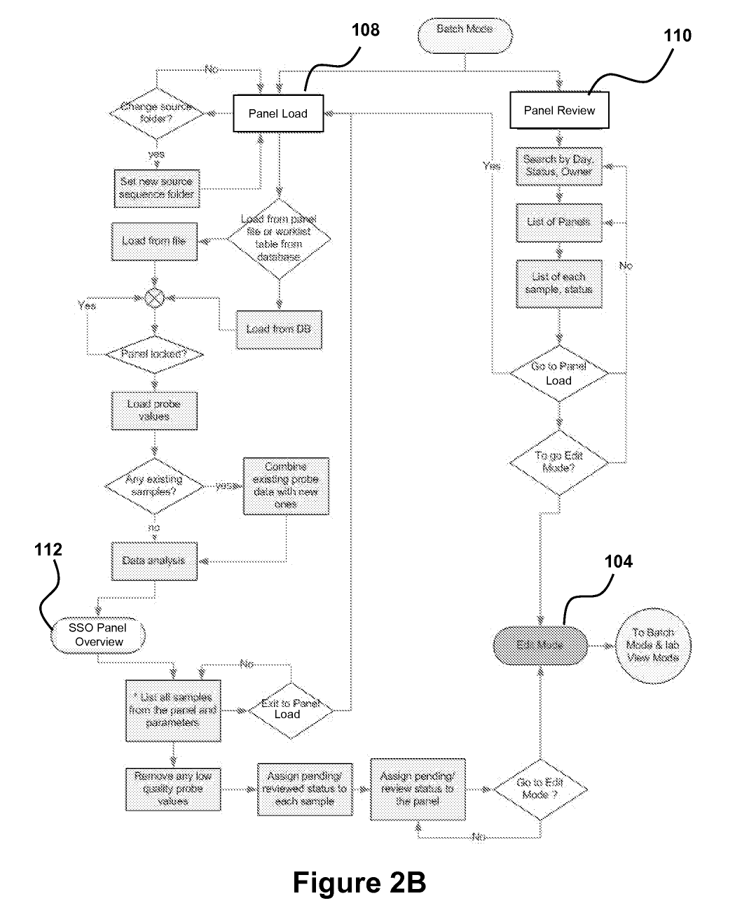

The super high throughput workflow (SHTP) 124, as depicted in FIG. 2A, starts from the step of loading a panel from a worklist input (stage 126). The samples are processed to get typing results. Any sample without a perfect match typing is designated as a failed sample. In one aspect of the invention, if the total number of failed samples is more than a user selected percentage of the number of samples in a given panel, the panel is rejected (stage 128). A lower throughput workflow may be employed to deal with those rejected panels.

The processing of the sequencing results is done in Batch Mode 102 (FIG. 2A refers to sequence generated worklists and FIG. 2B to probe (e.g., SSO) generated worklists) from the BM Panel Review 108 (FIG. 5), where the software system 100 evaluates the available sequence files on the network and assembles all files related to the samples in the Panel. Panel Load 108 also enables user ownership of the Panels which tracks review, approval, and turn around time metrics for all samples in the Panel in the Lab Mode 106, described in more detail below. A user may be tracked when logging on to the software. When a user loads a panel, the software checks if the panel is already loaded before. When a user saves a panel, the user name is saved along with the panel in the software's database. The user who submits a panel for approval and the user who approves a panel are also tracked with time stamp. Therefore, a fully approved panel may have three users associated, the original user who loads, a user who submits, where most of the time it is the same person as the original user but not necessarily, and the user who finally approves the panel. Each of those users may be assigned different authorizations. Another way to transfer the panel is from a LIMS to a shared database table. The shared database table is accessible to the said software system 100.

The panel is further processed 162 to gather samples which have ambiguity. Such ambiguity can be resolved by a Group Specific Sequence Primer (GSSP), as will be described in further detail below. The user then enters Edit Mode 104 for those failed samples, where manual editing is undertaken to determine if the result is a perfect match or to discard the failed sample.

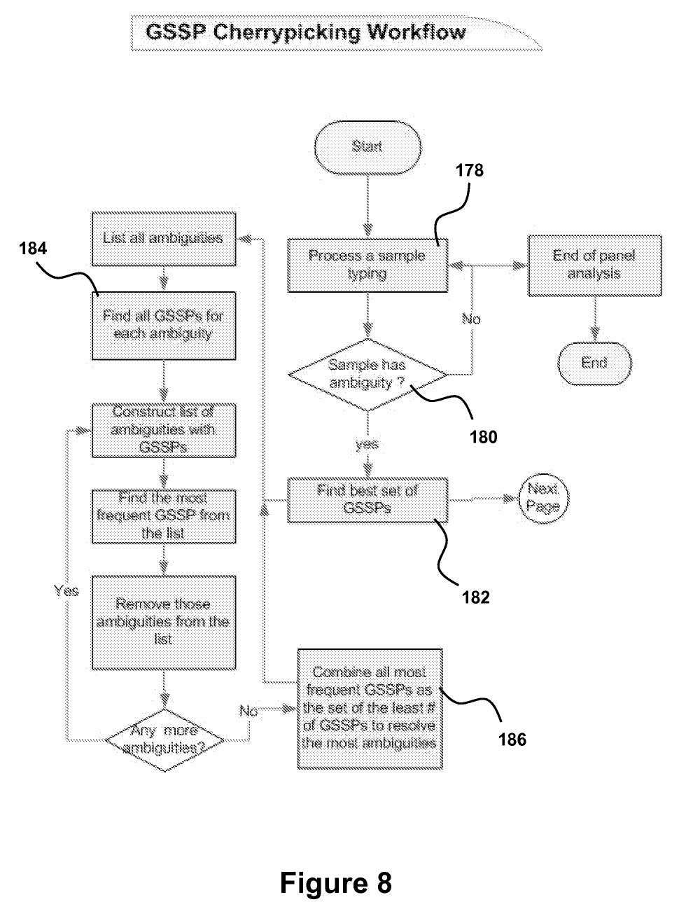

The samples that need GSSPs are then finally approved for a “cherry pick process” in a later stage (stage 136), that will be described in more detail below and in FIGS. 8-10. The dashed arrow of FIG. 2A indicates external steps to prepare GSSP panel/sample/sequencing before reanalysis with GSSPs data. The workflow described herein significantly decreases the time for data analysis per locus sample compared to conventional methods. For example, the time may be set according to metrics tracked by users logged onto the system.

The workflow for the panel load is depicted by the flow chart in FIG. 4. Any raw data files from any sub folders under the pre-designated location that are associated to a given sample from the panel will be loaded (stage 152) (e.g., sequencing or probing/hybridizing raw data) into the said software system 100. Further, if the sample has previously processed data (stage 158) which is stored in the software system's database and storage, the previously processed data must be loaded into the said software system (stage 160) to combine the new sequencing raw data files for data analysis (stage 162).

A sample said to have a complete set of sequence data is to have all necessary sequence data that can be obtained from available reagent products. For example, in HLA-specific typing Class I loci A, B, Cw typically have four or six sequences to cover exon 2, 3, or 4 in both directions. Class II DRB may only have 3 sequences, both directions for exon 2 and a sequence for codon 86.

The system will also further check if the panel is already being loaded or processed by other users. In one non-limiting example, the system is to designate a panel loaded but not submitted for review or approved with “owned” status. If the owner of a panel is different from the current user who loads it, the panel is deemed as “locked” (FIG. 4A, stage 154). It cannot be loaded by the current user. If the owner is the same, the user is still able to reload the panel.

As previously described above for FIG. 4, if some sequencing files for certain given samples have already been processed before (stage 158), those sequencing files are also loaded (stage 160). The samples then are processed accordingly (stage 162). Panel Overview 112 follows after the sequencing data analysis (stage 164).

After worklist loading of Panel Load 108, a panel load display, as shown in FIG. 6, is implemented to show the panel in a layout of wells 138, such as a 96 well tray or a 384 well tray, depending on the panel loaded. The list of sequencing data files can also further be displayed in the BM Panel Review 110. Additionally, a color-coded icon can be implemented to show if all necessary sequencing files are present for a specific sample. Such a panel display gives the user a quick assessment of the completion of a panel, meaning that sequencing raw data files are all loaded for a successful typing analysis. If various files are missing due to scenarios such as failed sequencing file output from a sequencer or networked storage failure, no further analysis is necessary on the loaded panel. The panel will be rejected without waste of further analysis time.

The Panel Review 110, shown and described in the workflow of FIG. 5, provides a way for a user, especially a supervisor, to review any panels that have been loaded, reviewed, and approved. In a minimal implementation, a user can search panels by day, status or ownership (stage 146). A user can select a panel for review, and have an option to go to Panel Overview 112 (stage 148) or go to Edit Mode 104 (stage 150) for further review in detail. One example in the selected panel on Panel Review 110 is to display the selected panel in a 96 well tray layout. Each well presents a sample from the panel. A status indicator like a color-coded icon is implemented to show if the sample has complete set of sequence data or requires further review.

Panel Review 110 displays all in-process panels in the workflow and users can track ownership, approval status, quality and productivity metrics for each panel. Clicking on the panel of interest launches Panel Overview 112 and loads all the output files for each sample in the Panel (e.g., sequencing files or probe files). Additionally, GSSP, SSP (“Specific Sequence Primer”) and non-SBT testing and data interpretation worklist outputs can be generated from Panel Overview for ambiguity resolution. For the DRB1 workflow, 40% of ambiguities can be resolved with a single GSSP targeting the codon 86 GTG sequence motif. More than two DRB1 panels can be processed before a full GSSP 96 well plate is at capacity.

From Panel Load 108 the analysis of genotypes is accessed through Overview 112, as shown in FIG. 7, by displaying the results from assembled sequences files for each sample. This includes genotype agreement with the positive control for batch process quality control; list of potential genotypes for each sample with the number of mismatches to known alleles; sequence quality metrics such as signal intensity and background; suggested follow-up testing reagents (GSSP/SSP/non-SBT testing and data interpretation (SSO)); basecalling discrepancies; and flags on samples that have potential significance. For the DRB1 workflow of one Panel, 190 ABI sequence files will be assembled and processed.

The Panel Overview 112, as shown in FIG. 7 and depicted in the flow chart of FIG. 3, provides a quick overview of the status for each sample in the panel by listing all samples from the panel and its quality parameters (stage 166). Parameters include, but are not limited, averaged signal of each type of base, noise to signal ratio, number of edits, number of differences in forward and reverse sequences, number of mismatches, typing and ambiguities, GSSP product codes for reducing ambiguities. A sequence of a sample can be manually removed from data analysis (stage 168). A status to a sample, such as pending for review, submitted for approval can be assigned to each sample (stage 170) or applied to the whole panel (stage 172). The user can go back to Panel Load (stage 174) or go into Edit Mode 104 (stage 176) for detailed data analysis such as sequence analysis, for example.

One non-limiting example, as illustrated in FIG. 7, is to list the typing results for each sample. Ambiguous typing results can be provided in an embedded dropdown list. Overall quality for a given sample also can be visually indicated by a color-coded item 122. For example, a sample with perfect match will have a full green dot 700, while a sample with no perfect match has a partial red/green dot 710. Also the visual Panel Overview can list the number of discrepancies between forward and reverse sequences, number of mismatched bases between consensus sequence and the pattern sequence. The pattern sequence is the sequence compiled from the sequencing raw data file. The consensus sequence is the reference sequence provided from database (such as an HLA alignment database for HLA genotyping). Certain bases with special base call methods from the said software system can also be shown.

Further, parameters such as quality parameters for each raw data file can also be displayed for review, either in a full display or a condensed view which is extendable to the full display. For example, for each sequencing raw data file, the averaged signal for each type of base (A, G, T, C) can be displayed. Other parameters such as noise to signal ratio can further indicate the quality of the sequencing data. Noise can be defined as the background peak height, while the signal is base peaks. Visual assistance such as icons can also be used to indicate if any parameter is within or outside a pre-determined range.

Further, a condensed display to show the sequencing trace curve can also be provided, along with trimmed area to give user a quick overview of the electropherogram of the sequencing data file. The trimmed area indicates the beginning or ending at a sequence that is not used for typing.

One innovation of Panel Overview 112 is the reduction in reviewing time, including a user's reviewing time. A user can quickly make the assessment of the panel and decide the next step. If there are many red dots on samples, indicating many samples having no perfect matches (failed sample), the next step will be to reject the panel. In one embodiment, if the percentage of samples having a perfect match (e.g., those tagged with a green dot) are greater than a user selected approval level, the next step is to submit the panel. The user may choose to review each failed sample on Edit View or confirm any GSSPs for ambiguity reduction before closing the panel.

Users tend to spend more time on a graphics data presentation than necessary. Without showing the electropherogram of sequencing data on a main window, the Panel Overview 112 bypasses the lengthy review of each sample. Instead, it gives an overview of key parameters to describe each sequence, each sample, and thus the whole panel. Significant time can be saved by introducing a summary view like Panel Overview 112 to give a quick assessment on a whole panel rather than focusing on individual samples to increase the data analysis throughput in a lab.

As shown in the Panel Overview flowchart in FIG. 3, from Panel Overview (with an exemplified embodiment of a displayed Panel Overview provided in FIG. 7) users can approve genotypes directly for one sample (stage 170) or the entire Panel (stage 172); reject results for one sample or the entire Panel; identify one or more samples for follow-up testing (stage 174); and launch into Edit Mode (stage 176) for individual raw sequencing data analysis and basecall editing. From Edit Mode users can return to Panel Overview and approve one or all samples in the Panel. Panel Overview then displays the number of edits and trim positions set by the user during Edit Mode.

In an aspect of the present invention, SHTP workflow makes it possible to significantly reduce the average analysis time used for each sample per locus. For example, for certain genotyping protocols, the average analysis time used for each sample per locus may be less than 3 minutes. To measure the time and thus improve the efficiency, the time starting from loading a panel is recorded. The sequence data analysis time, including basecalling and typing, is recorded. The time spent on manual editing and reviewing in Edit Mode if failed samples present and user chooses to do so is recorded. The panel submission for review and panel approval time is also recorded. The averaged time spent on each sample is the overall time spent on the panel as in the record divided by number of samples in the panel.

For comparison, obtaining approval time for similar genotyping procedures using only standard Edit Mode features approval time results in an increase in time to nearly 8 minutes per sample (e.g., 760 minutes for a Panel). This represents a 62.5% increase in genotyping turn around time. Accordingly, employing the processes disclosed herein can result in significant time savings, particularly for high-throughput labs handling many samples. Accordingly, one aspect of the invention provides a reduction in genotyping turn around time, such as an at least 30% reduction, at least 50% reduction, or at least 60% reduction compared to standard evaluation software.

Edit Mode

This mode offers features of a standard evaluation software. For example, in SBT evaluation where electropherograms can be viewed and base calls can be edited. Such features have been available for many years in software packages such as HLA Factura (Applied BioSystems). The main features allow loading of sequence files, alignment of the electropherograms, editing of base calls, sequence trimming, creation of a contiguous sequence from multiple overlapping sequence reads, and ultimately comparison to a database of known sequence types. The processes and systems provided herein are not restricted to any particular edit mode procedure or protocol, but instead may be tailored to provide output that is compatible with the Edit Mode software, as desired.

Secondary Evaluations

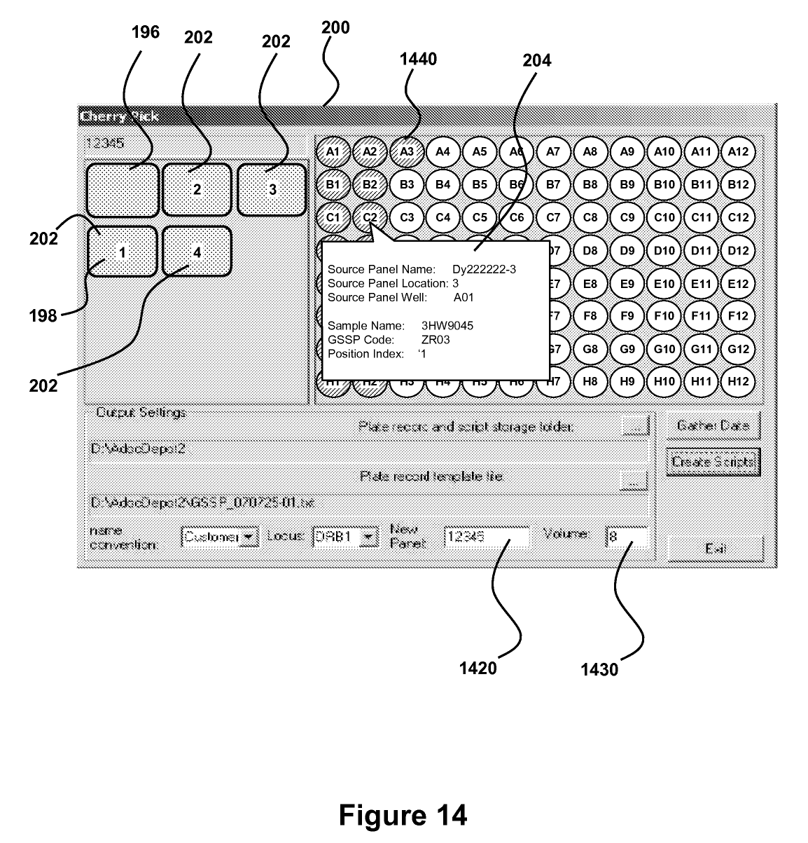

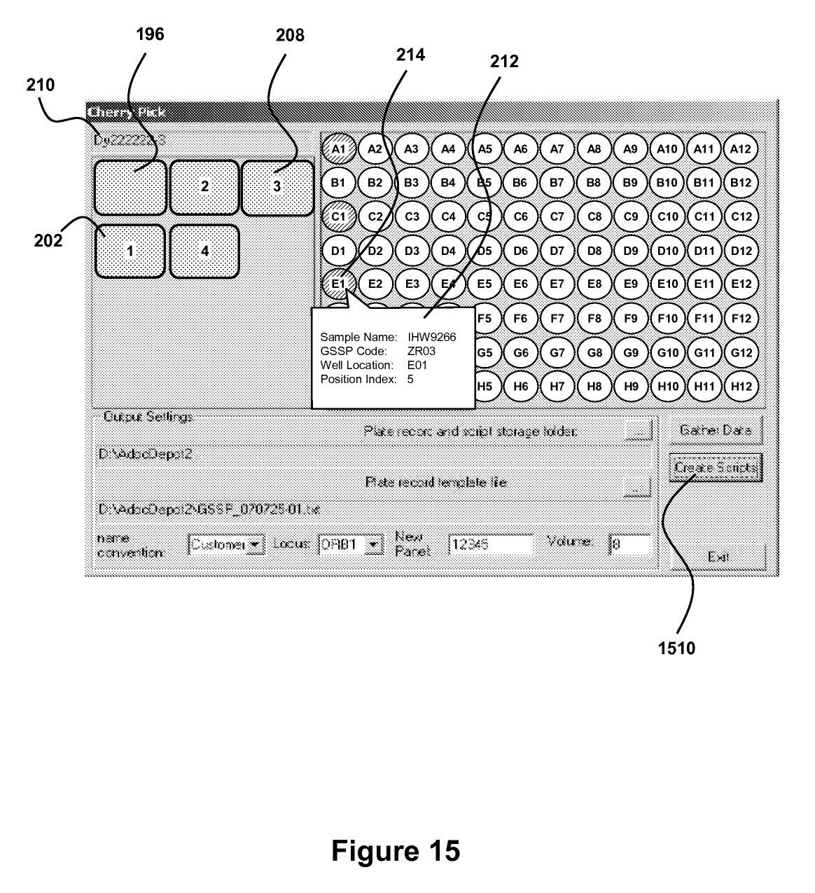

In an aspect of the present invention, an ambiguity resolution workflow to address the ambiguity reduction in a high-throughput typing environment is introduced, as illustrated by the “cherrypicking” workflow in FIGS. 8-10, and further shown by the illustrations in FIGS. 14-15. In an aspect, the ambiguity reduction is for high-throughput HLA typing. A typing ambiguity often results from the inability to determine the phase of two or more polymorphic positions in heterozygous sequence results. To resolve ambiguity, GSSPs sequences can be used because they produce sequence reads from only one allele and therefore elucidate the phase of multiple polymorphic positions. The process has two main steps. A first step is to generate the standard heterozygous sequences and analyze the typing results by comparing to the known alleles. A second step involves determining if any ambiguity exists and what GSSPs to use if such ambiguity can be resolved. If GSSPs are necessary, the GSSPs sequences are obtained. The GSSP sequencing typing results are combined with the regular sequencing typing results to get a final non-ambiguous HLA typing result. Further, a sample may have multiple ambiguities. Several GSSPs can be used to resolve those ambiguities. In an aspect, the software system will pick the least number of GSSPs to resolve the most ambiguities. This is one aspect that is said to make the present system and methods “efficient.”

A method of a software sub system accesses the said database and storage folders to calculate ambiguous genotype resolution reagents (ARR) to use for any sample that has multiple pairs of possible genotypes. A new worklist in a panel form and a script for liquid handler are created by the said software sub system. The said script can be used, for example, by a liquid handler for cherry picking samples from source panels to create the ARR sample panel for subsequent processes (e.g. sequencing, probing, hybridization, etc.). The script is further optimized for optimal sample processing, such as by minimizing the movement of a liquid handler.

Further, the use is optionally optimized so that least ARR will be used to resolve the most ambiguities in a given ARR panel.

The ARR worklist is then loaded by the said software system in when the ARR panel is sequenced. The said software system retrieves typing results and sequence data based on sample name and locus from the said database and storage folders. The retrieved samples are combined with the ARR data from the said ARR worklist to resolve any ambiguities. The results are stored in the said database and storage folders.

Further, a method of a software sub system accesses the said database and storage folders to calculate any SSP to use for any sample that has ambiguities after GSSP.

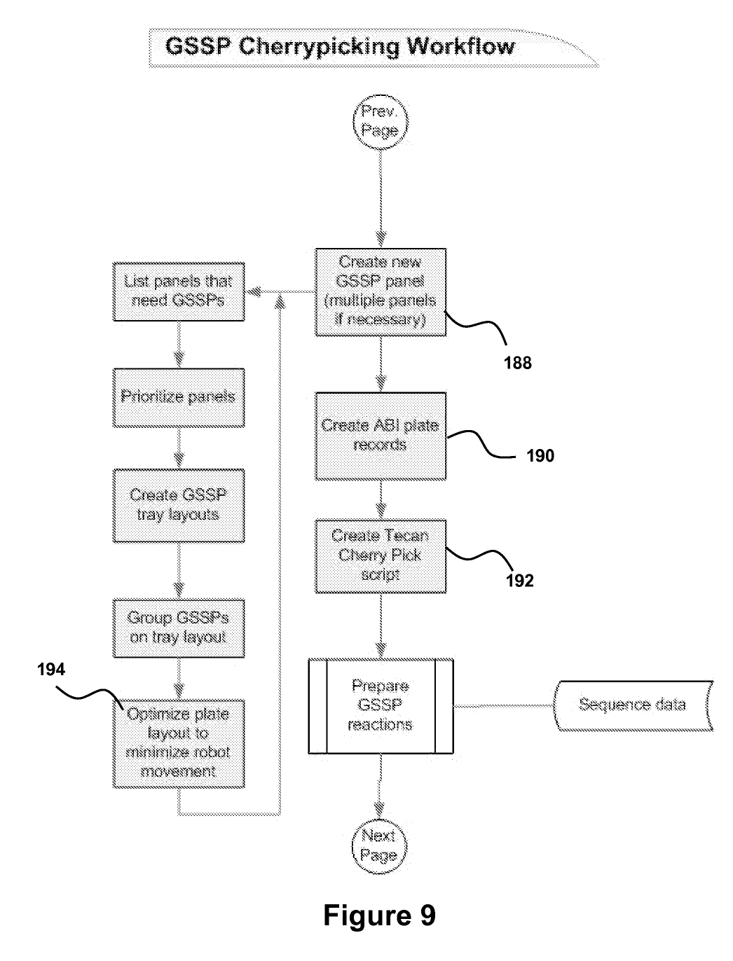

An example of the said workflow of the Cherry Pick Workflow implementation is illustrated in FIGS. 8-10. It starts from processing a sample typing (stage 178). If a sample has ambiguities (stage 180), GSSPs are obtained to resolve the ambiguities (stage 182). For a collection of samples, all GSSPs can be obtained (stage 184). The best set of GSSPs for the particular collection of samples can be achieved by finding the least number of GSSPs to resolve all ambiguities (stage 186). The new GSSP panel, or multiple panels if necessary, as shown in FIG. 9, is created (stage 188) along with plate record (stage 190) for sequencer such as ABI 3730xl and liquid handler script such as Tecan (stage 192). GSSP panel is created first by gathering data in which samples that need GSSPs to resolve ambiguity are compiled into such a way that the least movement of liquid handler head is resulted in (stage 194). Specifically, if a blue colored tray is designated as the destination tray 196 (see FIG. 14) (also referred to herein as a resolution panel or tray) which has the samples for GSSPs, all other trays contain the source samples which construct the destination panel. For example, Tray 1 198 has the most number of samples which need GSSPs that are used in the destination tray 196 and so is placed in a location adjacent the destination tray 196. The order for each tray, up to the maximum number of source trays that a given liquid handler can handle in one run, can be configured for optimal handling (e.g, the geometry that provides the least travel distance for a liquid handler), thereby decreasing processing time and increasing efficiency.

In the example shown in FIG. 14, the new panel 200 is a DRB1 locus panel with 17 samples that come from 4 source panels 202. The new panel name is assigned as “12345” 1420 and volume is set as desired (as indicated by the 8 uL entry 1430), as shown in FIG. 14. The highlighted tray on the upper left window is the colored in blue indicating it is the destination or “resolution panel” 196. The sample in a 96 well tray is also depicted in upper right window. Clicking on a well shows a sample information window 204, with information such as the source panel name, well, panel position in the liquid handler, GSSP code, sample name, and position index used by liquid handler.

As illustrated in FIG. 15, panels 202 (in green color) are source panels which have source samples that need to be cherry picked into the resolution (blue color) panel 196. The highlighted panel 208 is a source panel 202 currently selected for showing samples in the tray in detail. The panel name 210 is listed in the upper left corner and pop up message window 212 shows detail about the sample in the tray view. The green colored wells 214 are those samples that will be cherry picked into the destination panel 196.

Further, since the program will create a plate record for subsequent processes or tests to resolve ambiguities, (e.g., sequencing, SSO) and scripts for subsequent automated or robotic handling (e.g., a liquid handler), the destination folder to store these files is specified for subsequent use. A plate record template file is also preloaded to facilitate the creation of plate record. A Create Scripts 1510 button can accomplish the creation of both files as specified. Further, a layout print can also be generated for records. The print layout can help a user to set up the trays on a liquid handler.

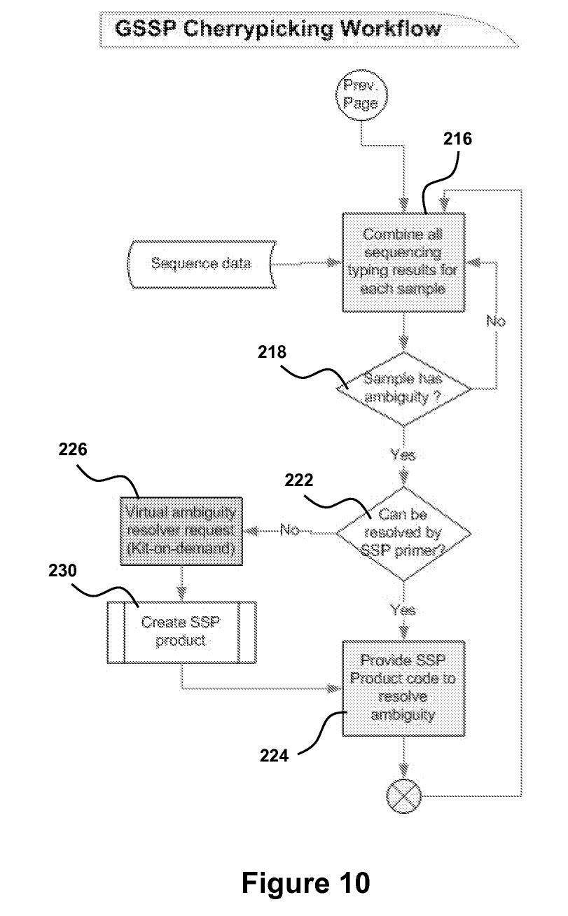

Even further, some ambiguities may remain even after apply GSSPs. A further ambiguity reduction workflow can be implemented, as illustrated by the flow chart in FIG. 10. Once the GSSP typing results are combined with the regular typing results (stage 216), if further ambiguity exists (stage 218), the ambiguities may be resolved by using SSP primer mix products (stage 222). If the SSP primer mix products exist, the product codes will be given (stage 224). One implementation could be that the said software system points to a product order form so that user can order the product directly from a reagent vendor if nothing is available on hand. Even further, if SSP is applied to the said sample that has ambiguity, the SSP result could be combined with sequencing results to reach the final non-ambiguous HLA typing result.

Yet another implementation is to provide a so called virtual ambiguity resolver, or kit-on-demand concept (stage 226). If SSP product is not available to resolve the ambiguity (stage 222), the key base or combined bases of several bases that can be used resolve the ambiguity can be calculated mathematically. That base or combined bases can be used to resolve the ambiguity in a virtual way. To really resolve the ambiguity, a primer is provided (stage 228) and a SSP kit constructed (stage 230) based on the key base or combined bases. This step can be implemented as a kit-on-demand process. In another implementation, the key base or combined bases can be provided to the user to be used for development of a “home-brew” reagent kit.

Lab Mode

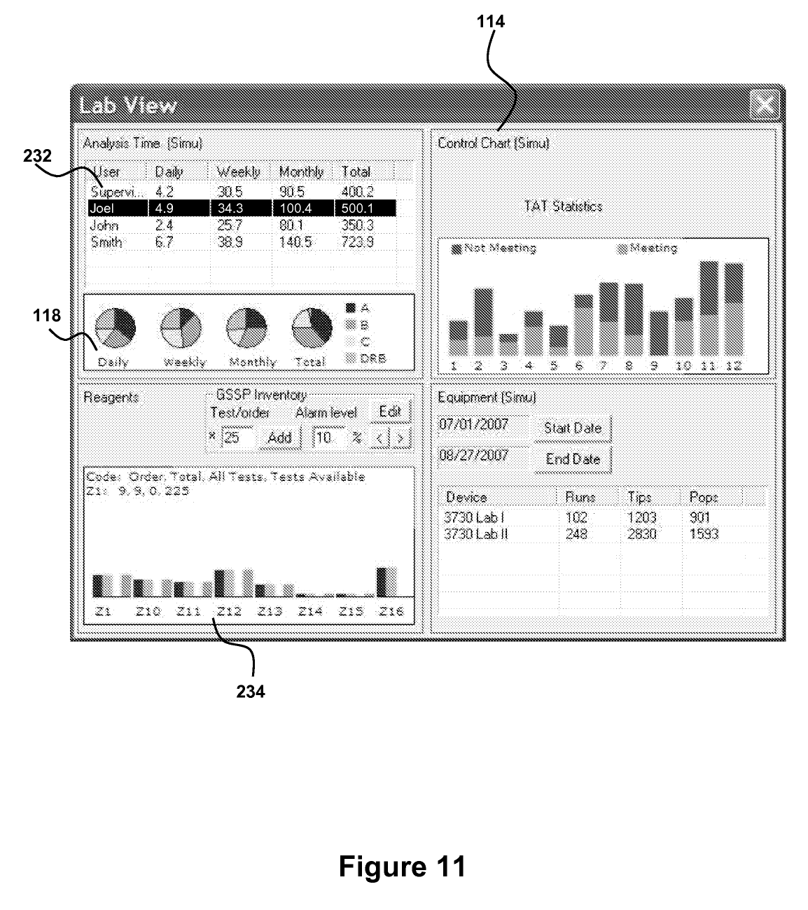

The final SW mode available is Lab Mode, shown in FIG. 11, where Supervisor users can produce outputs to monitor and track quality and productivity metrics. In the present invention Lab View provides a status overview of the typing activities in a given lab. It contains various views, especially, but not limited to, a control chart view 114, a SBT statistics view 116 (not shown in FIG. 11), and productivity view 118. While Panel Load, Panel Review, and Panel Overview in Batch Mode provides a workflow for genotyping such as high throughput HLA typing, Lab View provides a status check point for lab management to view the productivities and key statistics that can be used to pinpoint any area for efficiency improvement. One example is to list averaged or accumulated time spent on each locus, such as A, B, Cw, DRB, particular loci used in registry typing, given a specific period of time, as shown in the productivity view 118. A sum of the time from all users 232 also can be shown in productivity view. Such productivity view gives the lab supervisor or director a clear status report on the progress or area that needs attention. For example, if significant time spent on A than other loci in a given period of time, it may indicate problems in the reagents, conditions, or preparations of samples. The productivity view need not be used to gauge lab personnel productivity regarding job performance. Instead, the productivity view is useful for improving the efficiency of data processing and SBT high-throughput typing as a whole.

Further SBT statistics view can show reagents in hand and estimated demand 234, the sequencer's inventory and run parameters. The said functionalities can certainly be implemented in a LIMS or by other means. The innovation does not mean to limit to such implementation to the software system.

One of the key measurements for productivities is the turn around time (TAT) for a given period of time. At a preset threshold for meeting the targeted TAT value, TAT 236 can be charted as in meeting or not meeting the target at a monthly or another other chosen period of time. The Lab View can be displayed in a graphical way to better viewing, as illustrated in FIG. 11.

A method of a software sub system tracks SBT reagent inventory based on the estimate of sample usage. The inventory system further alarms users about the shortage of reagent inventory and may place reagent product order through print order, electronic ordering, or email. The inventory can be update and alarming levels can be set.

Further, a method of a software sub system tracks the time stamps on each sample and panel. The time during for analyzing a sample or a panel combined with the said parameters in (3) can be used to improve efficiency and productivity on person and laboratory.

A due date is further assigned to each panel. The said software system tracks the due date for each panel and gives warning to users if due date approaches to a preset date or period of time. The turn-around time can further be used to indicate productivity. The said warning may be a visual indicate or an email message.

A data mining method of a software sub system accesses the said database and storage folders to provide allele frequency information, haplotyping frequency information, based on population. Further, the need for ambiguity resolution product such as GSSPs or SSPs can be predicted based on the gathered information. Such predicted needs of products can be sent to reagent kit producers for better inventory control.

A data mining method of a software sub system accesses the said database and storage folders to provide sequence basecall quality information. The overall quality of sequence is tracked historically for the quality assurance.

User Access

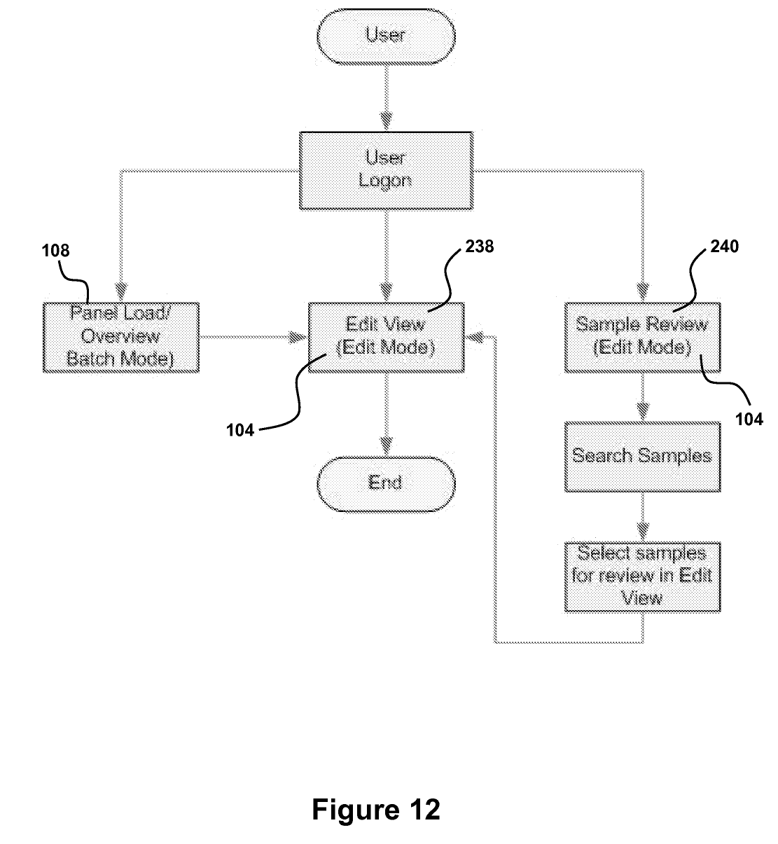

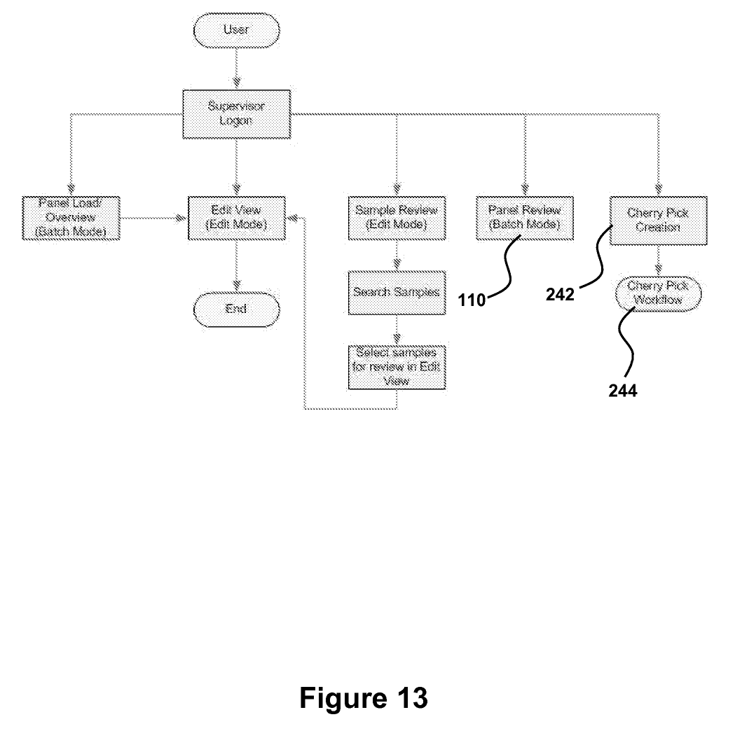

The SW offers one, two, or more than two, levels of access or authorizations: normal user, as illustrated by the workflow access in FIG. 12, and supervisor user, as illustrated by the workflow access in FIG. 13. This is to assure quality genotyping output by requiring two levels of review and approval before genotyping results are output to the LIMS for test fulfillment. For all Panels only a Supervisor User can approve genotypes for individual samples or the entire Panel. Users can Launch Edit Mode making use of fully functional sequence editing feature but then only recommend final approval.

As shown in FIG. 12, a regular user needs to logon first before use any features from the software system. The user can go to Panel Load 108 in Batch Mode, Edit View 238 in Edit Mode 104, or Sample Review 240 in Edit Mode 104. The sample review 240 in Edit Mode provides the user a way to retrieve processed sequencing sample data from either Batch Mode or Edit Mode. It has features for sample search and sample selection for review. A user can switch from different modes and views.

In FIG. 13, a supervisor user needs to logon first before use any features. In addition to the workflow as a regular user, a supervisor user also can access Panel Review 110 in Batch Mode and Cherry Pick Creation 242. In a different implementation, the Panel Review of Batch Mode and Cherry Pick Creation and Workflow 244 can also be accessible to a regular user through configurable software settings. A Cherry Pick Creation 242 and Workflow 244 feature provides a way to create a cherry pick process including making an efficient and economic arrangement of GSSP usage, creating a script for liquid handler control, and plate records for a sequencer.

The description of the present invention has been presented for purposes of illustration and description, but is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated. Moreover, although described above with respect to methods and systems, the need in the art may also be met with a computer program product containing instructions for modeling the error characteristics of a communications system.

It is important to note that while the present invention has been described in the context of a fully functioning data processing system, those of ordinary skill in the art will appreciate that the processes of the present invention are capable of being distributed in the form of a computer readable medium of instructions and a variety of forms and that the present invention applies regardless of the particular type of signal bearing media actually used to carry out the distribution. Examples of computer readable media include recordable-type media such as a floppy disk, a hard disk drive, a RAM, and CD-ROMs and transmission-type media such as digital and analog communication links.

Accordingly, the invention is to be defined not by the preceding illustrative description but instead by the spirit and scope of the following claims.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

Whenever a range is given in the specification, for example, a temperature range, a size range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting information or inputs, outputs, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method for evaluating the quality of a plurality of genotyping samples, the method comprising:
reviewing an interactive list of genotyping samples, wherein the interactive list comprises genotyping information and at least one selected quality parameter, and wherein the interactive list is displayed by a computer-program product embodied on a computer-usable medium; and
selecting a plurality of genotyping samples in the list as approved for further use, rejected from further use, or forwarded for further testing to better determine the genotype, the selection being dependent on at least one selected quality parameter.

2. The method of claim 1, wherein the genotype samples forwarded for further testing are samples with genotype ambiguities.

3. The method of claim 2, wherein the genotype ambiguities of the samples are resolved and the samples resubmitted for further evaluation.

4. The method of claim 3, wherein the samples with genotype ambiguities are resolved through the use of sequence-specific oligonucleotide typing.

5. The method of claim 3, wherein the samples with genotype ambiguities are resolved through the use of sequencing-based typing.

6. The method of claim 5 wherein the samples with genotype ambiguities are resolved through the use of one or more Group Specific Sequence Primers (GSSPs), one or more Specific Sequence Primers (SSPs) or both.

7. The method of claim 6, further comprising determining the type of GSSPs, SSPs or both to use such that the fewest number of GSSPs or SSPs will resolve the greatest number of ambiguities.

8. The method of claim 5, comprising further processing samples with at least one ambiguity with GSSP processing.

9. The method of claim 8, further processing samples with at least one ambiguity after GSSP processing through the use of one or more Specific Sequence Primer (SSP) primer.

10. The method of claim 8, further comprising processing the samples with at least one ambiguity using a virtual ambiguity resolver when a SSP primer is unavailable, such that the virtual ambiguity resolver generates a virtual result for resolving the at least one ambiguity.

11. The method of claim 10, further comprising constructing an SSP kit based on the virtual result of the virtual ambiguity resolver, such that the at least one ambiguity of the genotype sample can be resolved.

12. The method of claim 1, wherein the method is carried out on a computer-program product embodied in a computer-usable medium.

13. The method of claim 1, wherein the genotyping samples are sequence specific oligonucleotide samples.

14. The method of claim 1, wherein the genotyping samples are sequencing samples.

15. The method of claim 1 wherein the genotyping samples are those for immune system receptor genotyping, red blood cell antigen genotyping, bacterial species identification, virus genotyping, or metabolic factor genotyping.

16. The method of claim 1, wherein the genotyping samples are HLA genotyping samples.

17. A method for managing and evaluating genotyping data, the method comprising:
receiving a plurality of data from which genotype can be determined from a plurality of samples;
generating an input worklist from the plurality of data from a plurality of samples, wherein the worklist includes information identifying each sample;
processing the sample data to determine genotype and at least one quality parameter of the genotype determination; and
displaying a summary of at least one genotype and at least one quality parameter of the sample data of at least a portion of the plurality of samples for evaluation by a user.

18. The method of claim 17, wherein generating the worklist comprises importing a worklist.

19. The method of claim 18 wherein the worklist is imported from a Laboratory Information Management System (LIMS).

20. The method of claim 17, wherein determining the at least one quality parameter is a parameter selected from the group consisting of signal-to-noise ratio, basecall records, quality value of the typing result, and mis-matching counts.

21. The method of claim 20, wherein displaying a quality value of the typing result comprises displaying a genotyping ambiguity.

22. The method of claim 21, further comprising further processing of one or more samples displaying at least one ambiguity.

23. The method of claim 22, wherein the samples with genotype ambiguities are resolved through the use of sequence-specific oligonucleotide typing.

24. The method of claim 22, wherein the samples with genotype ambiguities are resolved through the use of sequencing-based typing.

25. The method of claim 24, wherein the samples with genotype ambiguities are resolved through the use of one or more Group Specific Sequence Primers (GSSPs), one or more Specific Sequence Primers (SSPs) or both.

26. The method of claim 25, further comprising determining the type of GSSPs, SSPs or both to use such that the fewest number of GSSPs or SSPs will resolve the greatest number of ambiguities.

27. The method of claim 25, comprising further processing samples with at least one ambiguity with GSSP processing.

28. The method of claim 27, further processing samples with at least one ambiguity after GSSP processing through the use of one or more Specific Sequence Primer (SSP) primer.

29. The method of claim 27, further comprising processing the samples with at least one ambiguity using a virtual ambiguity resolver when a SSP primer is unavailable, such that the virtual ambiguity resolver generates a virtual result for resolving the at least one ambiguity.

30. The method of claim 29, further comprising constructing an SSP kit based on the virtual result of the virtual ambiguity resolver, such that the at least one ambiguity of the genotype sample can be resolved.

31. The method of claim 17, wherein the method is carried out on a computer-program product embodied in a computer-usable medium.

32. The method of claim 17, wherein the data is sequence specific oligonucleotide data.

33. The method of claim 17, wherein the data is sequencing data.

34. The method of claim 17 wherein the genotype determined is that of an immune system receptor, red blood cell antigen, bacterial species, virus, or metabolic factor.

35. The method of claim 17, wherein the genotype determined is an HLA genotype.

36. The method of claim 17, further comprising measuring the time required for processing the genetic sequence data, analyzing the resulting typing and related information, and making a usability determination to look for problems, identify delays or both in the high-throughput genetic sequencing process.

37. A method for determining the quality of high-throughput data from which genotype can be determined of a plurality of samples, the method comprising:
processing data of a plurality of the samples to determine genotyping information and at least one quality parameter of the data;
displaying a summary of the genotyping information and the at least one quality parameter for at least a portion of the plurality of samples; and
analyzing the summary of the genotyping information and at least one quality parameter to determine the usability of the displayed samples for determining genotype at the same time point.

38. The method of claim 37, wherein the at least one quality parameter is selected from the group consisting of signal-to-noise ratio, basecall records, quality value of the typing result, and mis-matching counts.

39. The method of claim 37, wherein the at least one quality parameter is the identification of a genotype ambiguity.

40. The method of claim 39, further comprising resolving genetic ambiguities in the samples by forwarding samples with genotype ambiguities for further processing.

41. The method of claim 40, wherein the genotype ambiguities of the samples are resolved through the use of sequence-specific oligonucleotide typing.

42. The method of claim 40, wherein the samples with genotype ambiguities are resolved through the use of sequencing-based typing.

43. The method of claim 42, wherein the samples with genotype ambiguities are resolved through the use of one or more Group Specific Sequence Primers (GSSPs), one or more Specific Sequence Primers (SSPs) or both.

44. The method of claim 42, further comprising determining the type of GSSPs, SSPs or both to use such that the fewest number of GSSPs or SSPs will resolve the greatest number of ambiguities.

45. The method of claim 40, comprising further processing samples with at least one ambiguity with GSSP processing.

46. The method of claim 45, further processing samples with at least one ambiguity after GSSP processing through the use of one or more Specific Sequence Primer (SSP) primer.

47. The method of claim 40, further comprising processing the samples with at least one ambiguity using a virtual ambiguity resolver when a SSP primer is unavailable, such that the virtual ambiguity resolver generates a virtual result for resolving the at least one ambiguity.

48. The method of claim 47, further comprising constructing an SSP kit based on the virtual result of the virtual ambiguity resolver, such that the at least one ambiguity of the genotype sample can be resolved.

49. The method of claim 37, wherein the data comprises sequence specific oligonucleotide data, sequencing data or both.

50. The method of claim 37, wherein the data is sequencing data.

51. The method of claim 37, wherein the data is genotyping data for immune system receptor genotyping, red blood cell antigen genotyping, bacterial species identification, virus genotyping, or metabolic factor genotyping.

52. The method of claim 37, wherein the data is for HLA genotyping.

53. The method of claim 37, wherein the method is carried out on a computer-program product embodied in a computer-usable medium.

54. A computer-program product embodied on one or more computer-usable mediums for determining the quality of high-throughput data which can be used for determining genotype of a plurality of samples, and comprising computer instructions for:
processing the data for a plurality of samples to determine typing information and at least one quality parameter of the data; and
displaying the typing and at least one quality parameter for the plurality of samples in a single view, such that a user can analyze and make determinations of the usability of the samples for genotyping without requiring analysis of individual sample typing information.

55. The computer program product of claim 54, further comprising computer instructions for measuring the time required for processing the data, analyzing the resulting genotyping and at least one quality parameter, and determining usability to look for problems, identify delays or both in the high-throughput genetic sequencing process.

56. The computer program product of claim 55, further comprising computer instructions for further processing at least one sample selected by a user for further analysis; wherein the further processing comprises resolution of at least one genotype ambiguity.

57. The computer program product of claim 55, further comprising computer instructions for identifying one or more processes for resolving the ambiguity.

58. The computer program product of claim 57 wherein the processes for resolving the ambiguity comprise sequencing-based typing.

59. The computer program product of claim 57 wherein the processes for resolving the ambiguity comprise identifying one or more GSSPs for resolution of the ambiguity.

60. The computer program product of claim 57 wherein the processes for resolving the ambiguity comprises identifying one or more SSPs for resolution of the ambiguity.

61. The computer program product of claim 55, further comprising computer instructions for generating a script to provide instructions for further processing the one or more samples by the one or more identified processes for resolving the ambiguity.

62. The computer program product of claim 55, further comprising computer instructions for generating a script to provide instructions for further processing the one or more samples by the one or more identified processes for resolving the ambiguity.
wherein the script is for use by a liquid handler.
